(19) United States
(12) Patent Application Publication
(10) Pub. No.: US 2021/0102200 A1
(43) Pub. Date: Apr. 8, 2021

(54) SMALL MOLECULE TARGETED RECRUITMENT OF A NUCLEASE TO RNA

(71) Applicant: The Scripps Research Institute, La Jolla, CA (US)

(72) Inventor: Matthew D. Disney, Jupiter, FL (US)

(21) Appl. No.: 17/050,219

(22) PCT Filed: Apr. 24, 2019
(86) PCT No.: PCT/US2019/028955

§ 371 Date: Oct. 23, 2020

Related U.S. Application Data
(60) Provisional application No. 62/661,776, filed on Apr. 24, 2018.

(51) Int. Cl. C12N 15/11

(52) U.S. Cl. CPC C12N 15/11; C12N 2310/351

(57) ABSTRACT

Provided herein are compounds that selectively bind and cleave RNA. In various embodiments, the disclosure provides chemical compounds effective as ribonuclease targeting chimeras (RIBOTACs), that target the endogenous enzyme RNase L to selectively cleave the RNA in a living cell. These compounds are useful in the treatment of diseases, e.g., the treatment of breast cancer.

### CROSS-REFERENCE TO RELATED APPLICATION

This application claims the priority of U.S. provisional application Ser. No. 62/661,776, filed Apr. 24, 2018, the disclosure of which is incorporated herein by reference in its entirety

### STATEMENT OF GOVERNMENT SUPPORT

This invention was made with government support under GM097455 awarded by the National Institutes of Health. The government has certain rights in the invention.

### BACKGROUND

RNA drug targets are pervasive in all cells and in essentially all disease settings. The most common way to target RNA is with oligonucleotide-based modalities that bind complementary sequences largely in unstructured region. The resulting oligonucleotide:RNA hybrid recruits endogenous ribonuclease H (RNase H), which then cleaves the RNA target and affects biology. Oligonucleotides have been transformative medicines; however, they have platform-specific toxicities when delivered peripherally, such as thrombocytopenia in man. Small molecules can be an alternative approach to target RNA as they have historically been lead medicines and their chemical matter can be broadly medicinally optimized. Human RNA, however, is thought to be recalcitrant to small molecule targeting and as such is classified as undruggable. Obtaining bioactive small molecules targeting human RNAs is challenging and thus general solutions to this complex molecular recognition problem requires new approaches.

### SUMMARY

The disclosure provides, in various embodiments, chemical compounds effective as ribonuclease targeting chimeras (RIBOTACs), that target the endogenous enzyme RNase L to selectively cleave the primary transcript (pri-miR-96) of micro-RNA 96 (miR-96) in a living mammalian cell. Destruction of pri-miR-96 can selectively inhibit biogenesis of miR-96, thereby de-repressing the pro-apoptotic transcription factor FOXO1 (a downstream target of miR-96). Activation of the pro-apoptotic FOXO1 can trigger apoptosis selectively in triple negative breast cancer cells relative to normal breast cells.

In some aspects, the disclosure provides compounds of Formula I:

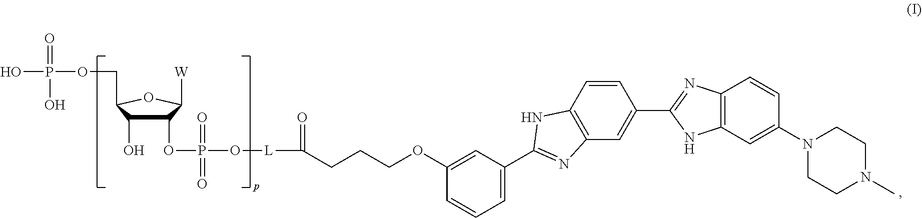

wherein W is a nucleobase, L is a linker moiety, R¹, R², R³, and R⁴ are each individually H or C₁₋₆alkyl, n is 0 to 9, o is 1 to 5, and p is 1 to 5. In various embodiments, L is a linker moiety having a structure

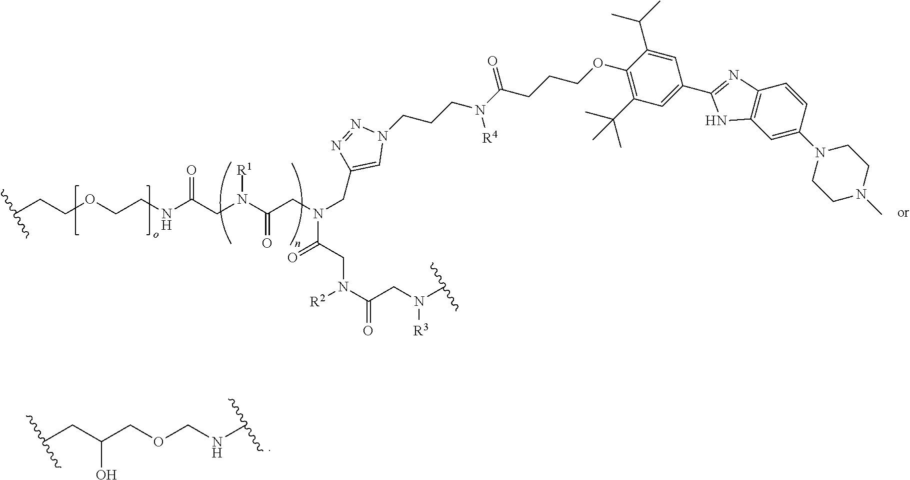

In various embodiments, the disclosure provides a compound of formula Ia:

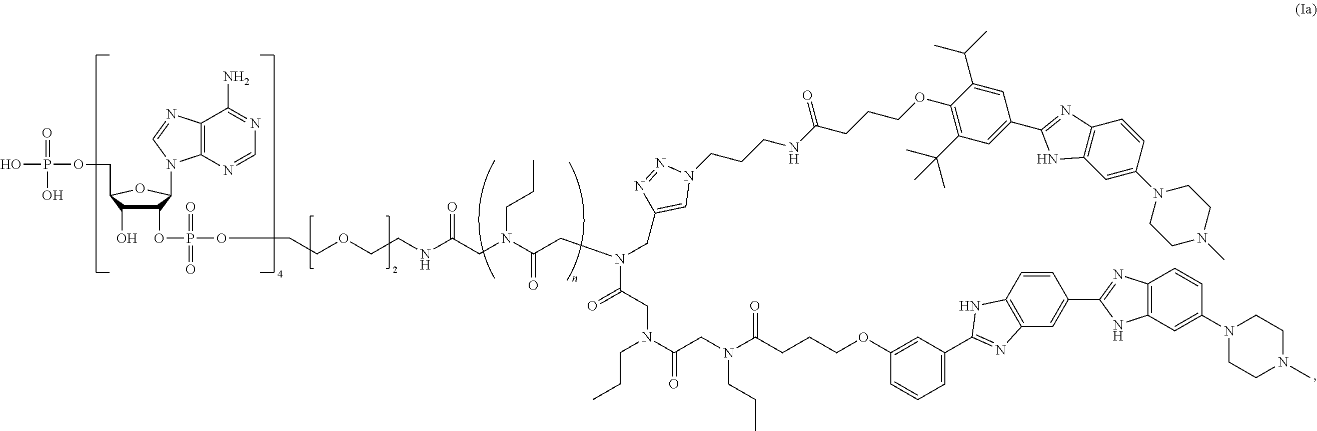

wherein n is 0, 1, 2, 3, 4, 5, 6, 7, 8, or 9; or a pharmaceutically acceptable salt thereof. The compound of this structure wherein n=0, was found to be particularly active in inhibiting biogenesis of miR-96, de-repression of FOXO1, and induction of apoptosis in breast cancer cells.

Accordingly, provided in various embodiments are methods of selectively cleaving an miR-96 precursor hairpin RNA, comprising contacting the miR-96 precursor hairpin RNA in a living cell with an effective amount or concentration of a compound disclosed herein. The living cell can be a cancer cell, such as a breast cancer cell.

Further, provided in various embodiments are methods of de-repressing pro-apoptotic FOXO1 transcription factor and triggering apoptosis in a breast cancer cell, comprising contacting the cancer cell with an effective amount or concentration of a compound as shown above, e.g., with any one of compounds 2, 3, or 4. The biological effect of a compound described herein, such as any of compounds 2, 3, or 4, that is administered in an effective amount or concentration of the compound sufficient to trigger apoptosis in a breast cancer cell, can be ineffective to trigger apoptosis in a healthy breast cell, providing a selective apoptotic effect versus cancer cells.

Further, the disclosure provides, in various embodiments, chemical compounds effective as RIBOTACs that target the endogenous enzyme RNase L to selectively cleave the precursor of miR-210, or pre-miR-210, in a living mammalian cell. Destruction of pre-miR-210 can selectively inhibit biogenesis of miR-210, thereby de-repressing the glycerol-3-phosphate dehydrogenase 1-like (GPD1L) protein, which binds to prolyl hydroxylase (PHD) to promote hyperhydroxylation of hypoxia inducible factor 1-alpha (HIF1α), mediating HIF1α degradation by the proteasome, and triggering apoptosis in a breast cancer cell. Activation of GPD1L can trigger apoptosis selectively in triple negative breast cancer cells relative to normal breast cells.

In various embodiments, the disclosure provides a compound of formula (TGP-210-RL) or a pharmaceutically acceptable salt thereof. The compound of this structure was found to be particularly active in inhibiting biogenesis of miR-210, de-repression of GPDL1, and induction of apoptosis in breast cancer cells.

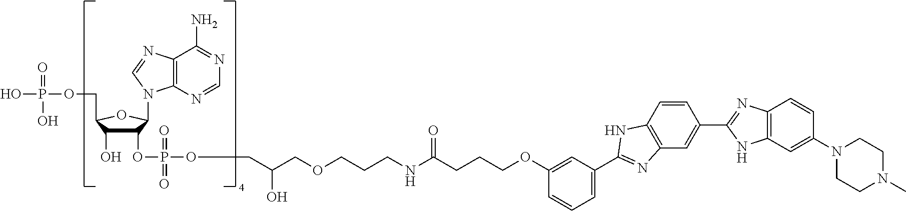

Accordingly, provided in various embodiments are methods of selectively cleaving pre-miR-210, comprising contacting pre-miR-210 RNA in a living cell with an effective amount or concentration of a compound disclosed herein. The living cell can be a cancer cell, such as a breast cancer cell.

Further, provided in various embodiments are methods of de-repressing GPDL1 and triggering apoptosis in a breast cancer cell, comprising contacting the cancer cell with an effective amount or concentration of a compound as shown above, e.g., with any one of compounds as disclosed herein, as listed in Table A, below, e.g., 1,2, 3, TGP-210-2′-5′ A₂, TGP-210-2′-5′ A₃, or TGP-210-2′-5′ A₄. The biological effect of a compound described herein, such as any of compounds as disclosed herein, as listed in Table A, e.g., 1,2, 3, TGP-210-2′-5′ A₂, TGP-210-2′-5′ A₃, or TGP-210-2′-5′ A₄, that is administered in an effective amount or concentration of the compound sufficient to trigger apoptosis in a breast cancer cell, can be ineffective to trigger apoptosis in a healthy breast cell, providing a selective apoptotic effect versus cancer cells.

Consequently, the disclosure provides methods, in various embodiments, of treating cancer such as breast cancer in a patient afflicted therewith. More specifically, the breast cancer can be triple negative breast cancer.

### BRIEF DESCRIPTION OF THE FIGURES

FIG. 1: Design and characterization of a transcript-selective RNase L recruiting compound. (A) Top, secondary structure of the primary transcript of microRNA-96 (pri-miR-96). Bottom, Schematic depiction of active RNase L recruitment through 2′-5′ A to pri-miR-96 by compound 2. (B) Compounds used in this study. (C) In vitro cleavage of pri-miR-96 by 2′-5′ A₄ and chimeric small molecules with different linker lengths; n=0 linker (2) displayed the highest cleavage capability. (D) Representative Western blot and quantification of cross-linked monomer and oligomer (active) forms of RNase L with treatment of 2′-5′A₄, 2 which is selective for pri-miR-96, or 1b which lacks 2′-5′A₄. Data are expressed as mean±s.e.m. (n≥3). *p<0.05, as measured by a two-tailed Student t test.

FIG. 2. Small molecule RNase L recruitment shows on-target effects in cells. (A) Treatment of MDA-MB-231 triple negative breast cancer cells with 2 decreased abundance of pri-miR-96 via cleavage, as compared to 1a, which boosted levels of pri-miR-96 by inhibiting Drosha processing, as measured by RT-qPCR. (B) Effect of 1a and 2 on mature miR-96 levels. (C) 2-mediated cleavage of pri-miR-96 is reduced by addition of 1a. Relative cleavage controls for the effect of 1a at the same concentration used for 2. (D) RT-qPCR of RNAs isolated from immunoprecipitated RNase L protein with 2′-5′A₄ or 2 treatment at 200 nM. RNAs bound to RNase L treated with 2 show enrichment of the pri-miR-96 transcript normalized to RNA immunoprecipitated from β-actin. (E) Relative cleavage of pri-miR-96 by 2 upon knock down of RNase L by siRNA. (F) RNase L overexpression resulted in increased cleavage activity of 2 (20 nM), while overexpression of pri-miR-96 resulted in decreased cleavage activity. Data are expressed as mean±s.e.m. (n≥3). *p<0.05, **p<0.01, as measured by a two-tailed Student t test.

FIG. 3. Apoptotic stimulation through selective recruitment of RNase L to pri-miR-96 by 2. (A) FOXO1 is a tumor suppressor protein down-regulated by miR-96. Treatment with 2 caused a de-repression of FOXO1 as measured by Western blot. (B) Selective cleavage of miR-96 with 2 treatment (20 nM), amongst predicted FOXO1-targeting miRNAs. (C) Non-hypothesis driven RT-qPCR analysis of validated miRNAs indicated inhibition of miR-96 by 2 (200 nM) with the most magnitude and significance. (D) Treatment with 2 (200 nM) caused MDA-MB-231 cells to become apoptotic as measured by Annexin V/PI staining. “Pri-miR-96” indicates plasmid overexpression of a pri-miR-96 hairpin, which diminished apoptosis caused by 2. Data are expressed as mean±s.e.m. (n≥3). *p<0.05, as measured by a two-tailed Student's t test.

FIG. 4 shows the synthetic route for preparation of compound 1b.

FIG. 5 shows the synthetic route for preparation of compound 3b.

FIG. 6 shows the synthetic route for preparation of compound 4b.

FIG. 7 shows the synthetic route for preparation of compound 2.

FIG. 8 shows the synthetic route for preparation of compound 3.

FIG. 9 shows the synthetic route for preparation of compound 4.

FIG. 10 shows the GPD1L protein binds to prolyl hydroxylase (PHD) to promote hyperhydroxylation of hypoxia inducible factor 1-alpha (HIF1α), thus mediating the polyubiquitination and subsequent degradation of this protein by the proteasome.

FIG. 11 shows that small molecule TGP-210 targets the Dicer site in pre-miR-210 and inhibits its Dicer processing, thus decreasing the biogenesis of mature miR-210-3p SEQ ID NO:1

FIG. 12 shows the structure of TGP-210-RL.

FIG. 13 shows that TGP-210-RL had the most potent cleavage effect, while very limited cleavage activity was observed for the TGP-210 derivatives appended with the dimer and trimer 2′-5′ oligoadenylates.

FIG. 14 shows a study of the binding consequences of adding the 2′-5′ A₄ nuclease recruiting module, with binding affinities measured by microscale thermophoresis (MST) to these targets with TGP-210-RL.

FIG. 15 shows in vitro RNase L dimerization, binding selectivity, and cleavage of pre-miR-210 by TGP-210-RL. (A) Representative Western blot and quantification of cross-linked monomer and oligomer (active) forms of RNase L upon treatment with 2′-5′ A₄, TGP-210-RL, and parent compound TGP-210. (B) Representative binding isotherms of TGP-210, TGP-210-RL, or TGP-210-RL with RNase L (50 nM) to 5′ Cy5 end labeled miR-210 Hairpin RNA by MST analysis. Green box indicates TGP-210/TGP-210-RL binding site on the miR-210 Hairpin RNA. (C) Representative binding isotherms of TGP-210, TGP-210-RL, or TGP-210-RL with RNase L (50 nM) to 5′ Cy5 end labeled miR-210 Mutant RNA by MST analysis. Orange box indicates the mutated binding site in the miR-210 Mutant RNA, which is the corresponding base paired control to the miR-210 Hairpin RNA. (D) Representative binding isotherms of TGP-210, TGP-210-RL, or TGP-210-RL with RNase L (50 nM) to 5′ Cy5 end labeled DNA Hairpin by MST analysis. (E) In vitro mapping gel of 5′ ³²P end labeled miR-210 Hairpin Precursor with RNase L and TGP-210-RL treatment. “OH” indicates hydrolysis ladder, which cleaves at every base; “T1” indicates denaturing cleavage conditions with T1 endonuclease that cleaves after every G base.

FIG. 16 shows compound cellular uptake and cleavage in MDA-MB-231 cells. (A) Left, relative cellular uptake of 1 μM of TGP-210 or TGP-210-RL in MDA-MB-231 cells, as measured by flow cytometry. Cellular uptake was detected via intrinsic compound fluorescence, upon excitation with a DAPI-UV Laser (Ex: 345 nm; Em: 460 nm). Right, representative overlaid flow cytometry histograms indicating positive counts of compound detection in cells. Signal from untreated and TGP-210-treated cells were normalized to 0% and 100%, respectively. (B) Confocal microscopy of 5 μM TGP-210 or TGP-210-RL-treated MDA-MB-231 cells stained with SYTO 82 Orange Fluorescent Nuclear Stain at 40× magnification. White scale bars represent 20 μm. (C) Relative cleavage of pre-miR-210 in hypoxic MDA-MB-231 cells by TGP-210 appended with different lengths of 2′-5′ Aₙ (n=2−4). (D) Transfection of 2′-5′ A₄ into hypoxic MDA-MB-231 cells does not significantly affect mature miR-210-3p and pre-miR-210 abundance, as measured by RT-qPCR analysis. *p<0.05, ***p<0.001, as determined by a two-tailed Student t test. Data represent means±SEM of triplicates.

FIG. 17 shows the selectivity of TGP-210-RL by RNA-Seq and effect of miR-210 targeting compounds on apoptosis in normoxic MDA-MB-231 cells. (A) RNA-Seq was performed on total RNA from vehicle (DMSO) or TGP-210-RL-treated (200 nM) hypoxic MDA-MB-231 cells after 24 h of treatment. Differential gene expression between the samples was plotted as a scatter plot of scaled reads per base of genes in vehicle samples (x-axis) and scaled reads per base of genes in TGP-210-RL-treated samples (y-axis). Out of 18829 mapped genes, no genes were significantly affected, with a false discovery rate of 1%, demonstrating that the compound has limited off-target effects. (B) TargetScanHuman v7.2 was used to predict the target genes of miR-210-3p only with conserved sites. Out of 42 predicted target genes, 37 genes were mapped to the RNA-Seq dataset. Relative % fold change indicated that 27 out of 37 target genes were upregulated, which indicates a significant discrepancy from a binomial distribution with a positive bias with 99% confidence, according to a binomial statistics test (>26, or >70%, upregulated targets represents discrepancy from a binomial distribution with 99% confidence). (C) TargetScanHuman v7.2 was used to predict the top 100 target genes of miR-23-3p, irrespective of site conservation, ranked by cumulative weighted context++ score. miR-23-3p was used as a control miRNA, since it is more highly expressed than miR-210-3p and is also a hypoxia-associated miRNA. Out of 100 predicted target genes, 80 genes were mapped to the RNA-Seq dataset. Relative % fold change indicated that 46 out of 80 target genes were upregulated, which obeys a binomial distribution, according to a binomial statistics test (>51, or >63%, upregulated targets represents discrepancy from a binomial distribution with 99% confidence). (D) TargetScanHuman v7.2 was used to predict the top 100 target genes of miR-107, irrespective of site conservation, ranked by cumulative weighted context++ score. miR-107 was used as a control miRNA, since it expressed at similar levels of miR-210-3p and is also a hypoxia-associated miRNA. Out of 100 predicted target genes, 96 genes were mapped to the RNA-Seq dataset. Relative % fold change indicated that 55 out of 96 target genes were upregulated, which obeys a binomial distribution, according to a binomial statistics test. (>59, or 61%, upregulated targets represents discrepancy from a binomial distribution with 99% confidence). Red lines indicate the 99% confidence interval for the fold change distribution to violate a binomial distribution. (E) MDA-MB-231 cells in normoxic conditions were treated for 48 h with a scrambled locked nucleic acid (Scr-LNA), a miR-210-3p LNA (LNA-210), TGP-210, and TGP-210-RL and the apoptotic stimulation was measured by Caspase 3/7 activity. Alternatively, normoxic MDA-MB-231 cells were transfected with a plasmid that overexpresses the miR-210 precursor (yellow box; Pre-miR-210 Overexpression) and treated as described above.

### DETAILED DESCRIPTION

Provided herein are compounds that can bind to and selectively cleave RNA in order to treat or prevent a disease or disorder. These compounds are useful in the treatment of a variety of diseases and disorders, including cancer e.g., breast cancer, or triple negative breast cancer.

### Compounds of the Disclosure

The disclosure provides compounds of Formula I and pharmaceutically acceptable salts thereof:

### Formula I:

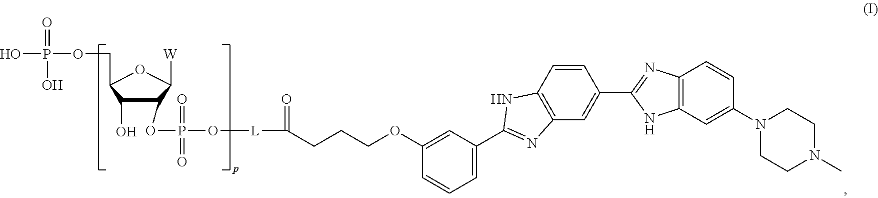

wherein W is a nucleobase; L is a linker moiety, and p is 1 to 5.

In some embodiments, each W is adenine or guanine. In some embodiments, each W is adenine.

In some embodiments, L comprises C₂₋₆alkylene-O—C₂₋₆alkylene-NR³— or

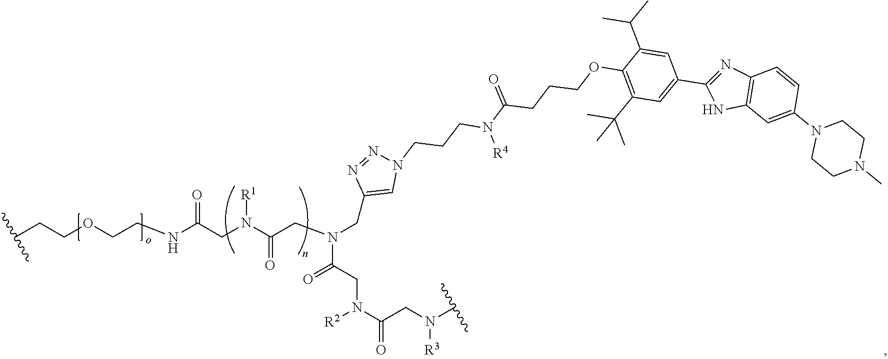

each C₂₋₆alkylene is optionally substituted with 1 or 2 OH, R¹, R², R³, and R⁴ are each individually H or C₁₋₆alkyl; n is 0 to 9; and o is 1 to 5.

In some embodiments, L is C₂₋₆alkylene-O—C₂₋₆alkylene-NR³. In some embodiments, L is C₂₋₄alkylene-O—C₂₋₄alkylene-NR³. In some embodiments, L is C₃alkylene-O—C₃alkylene-NR³. In some embodiments, L is C₂₋₆alkylene-O13 C₂₋₆alkylene-NR³ and is optionally substituted with 1 OH. In some embodiments, L is C₂₋₄alkylene-O—C₂₋₄alkylene-NR³ and is optionally substituted with 1 OH. In some embodiments, L is C₃alkylene-O—C₃alkylene-NR³ and is optionally substituted with 1 OH.

In some embodiments, L is

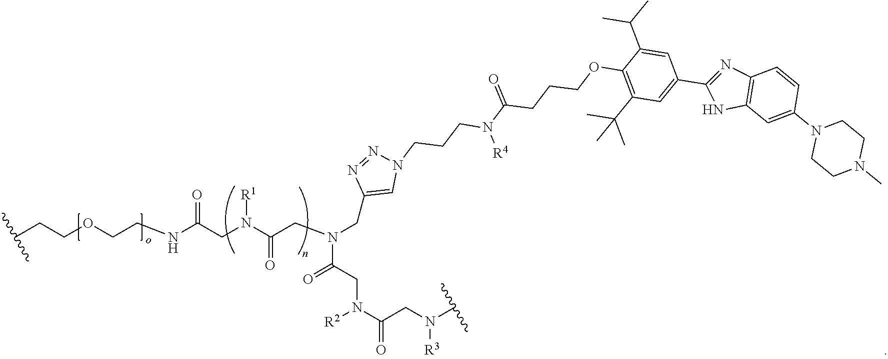

In some embodiments, R¹ is H. In some embodiments, R¹ is C₁₋₆alkyl. In some embodiments, R¹ is C₃alkyl. In some embodiments, R² is H. In some embodiments, R² is C₁₋₆alkyl. In some embodiments, R² is C₃alkyl. In some embodiments, R³ is H. In some embodiments, R³ is C₁₋₆alkyl. In some embodiments, R³ is C₃alkyl. In some embodiments, R⁴ is H. In some embodiments, R⁴ is C₁₋₆alkyl. In some embodiments, R⁴ is C₃alkyl.

In some cases, each R¹, R², R³, and R⁴ is H or C₃alkyl.

In some embodiments, L is

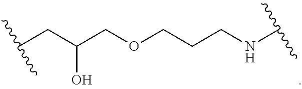 [SMILES: *CC(O)COCCCN*]

In some embodiments, L is

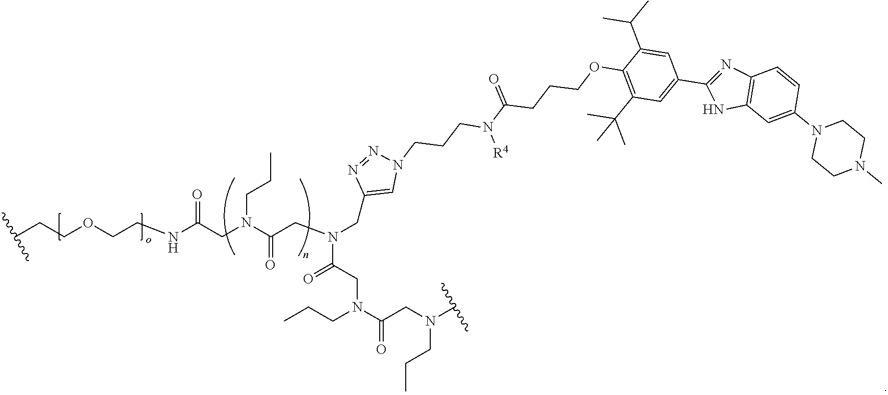

In some embodiments, p is 2, 3, or 4. In some embodiments, p is 1. In some embodiments, p is 2. In some embodiments, p is 3. In some embodiments, p is 4.

In some embodiments, o is 1, 2, 3, or 4. In some embodiments, o is 4. In some embodiments, o is 1 or 2. In some embodiments, o is 1. In some embodiments, o is 2. In some embodiments, o is 3. In some embodiments, o is 4.

Also provided herein are compounds of Formula (Ia):

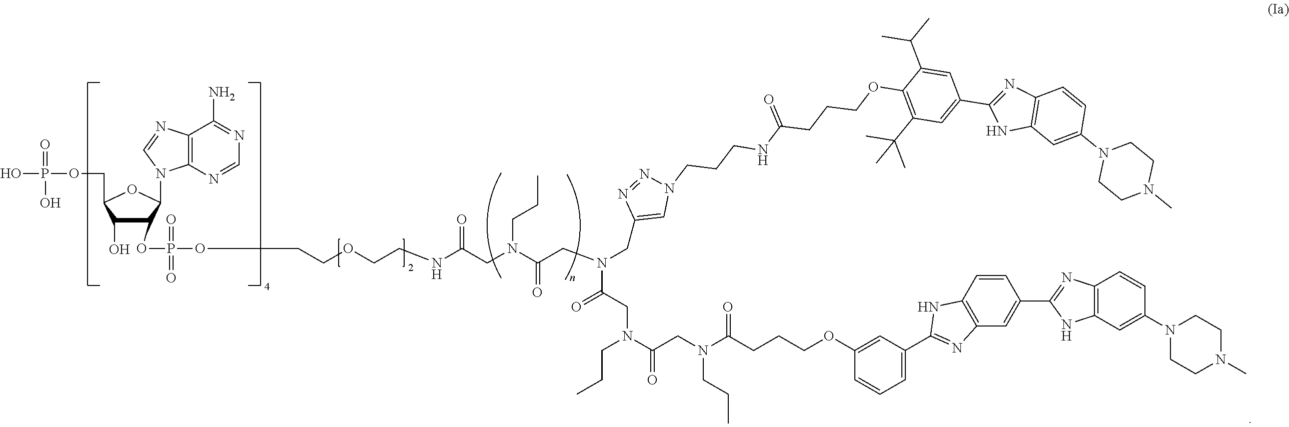

In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 0, 3, 6, or 9.

Particular compounds contemplated include those listed in Table A, below, and pharmaceutically acceptable salts thereof.

TABLE A

| Compound | |
|---|---|
| No. | Structure |
| 2 | 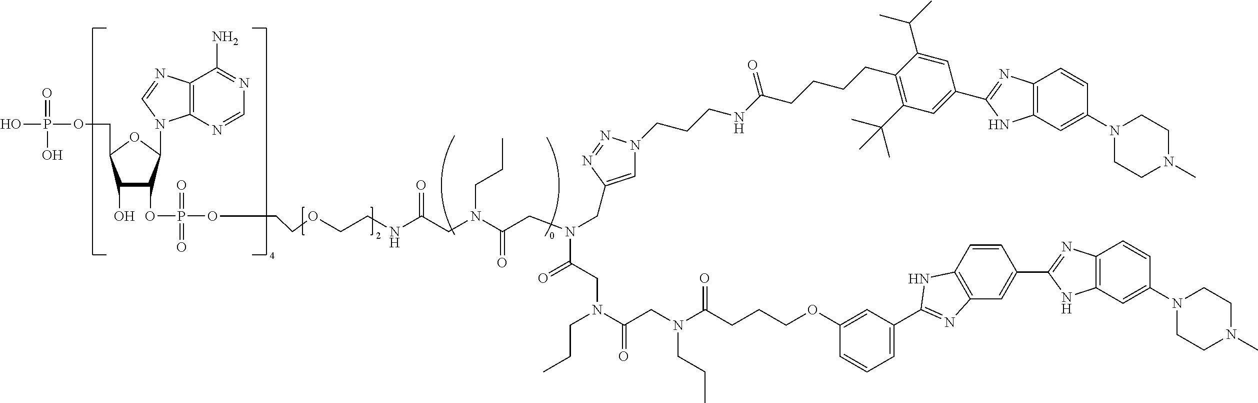 |
| 3 | 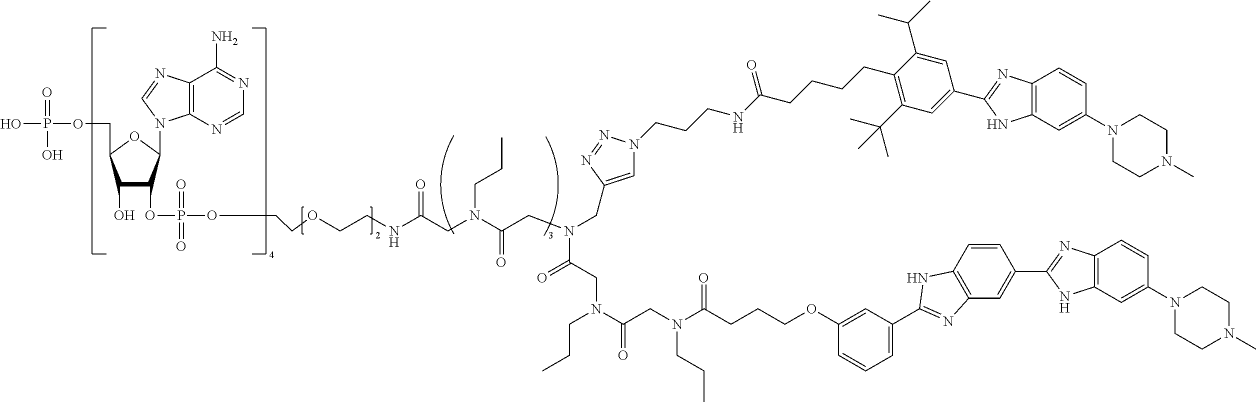 |
| 4 | 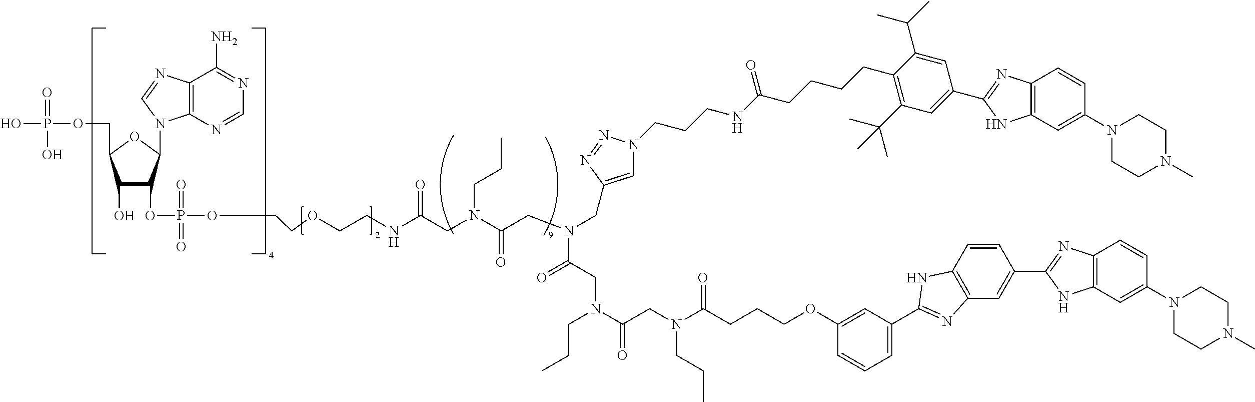 |
| TGP-210- 2′-5′ A₂ | 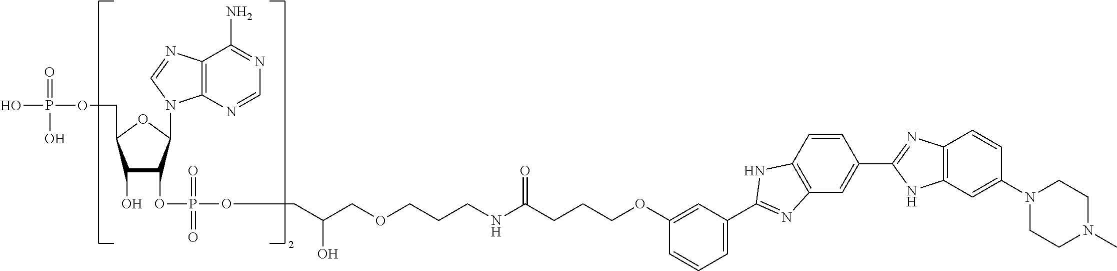 |
| TGP-210- 2′-5′ A₃ | 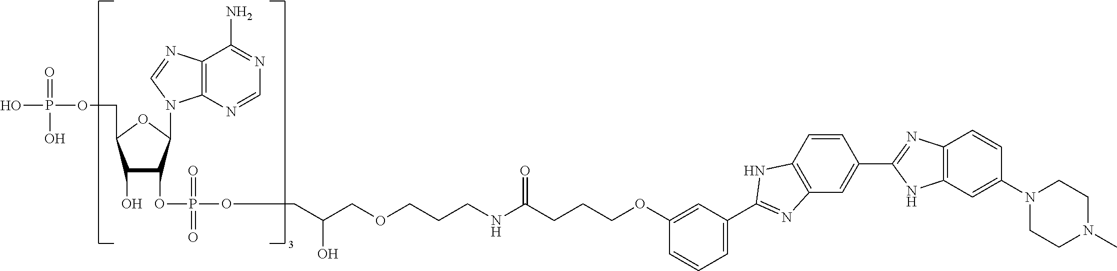 |
| TGP-210- RL (also called TGP-210- 2′-5′ A₄) | 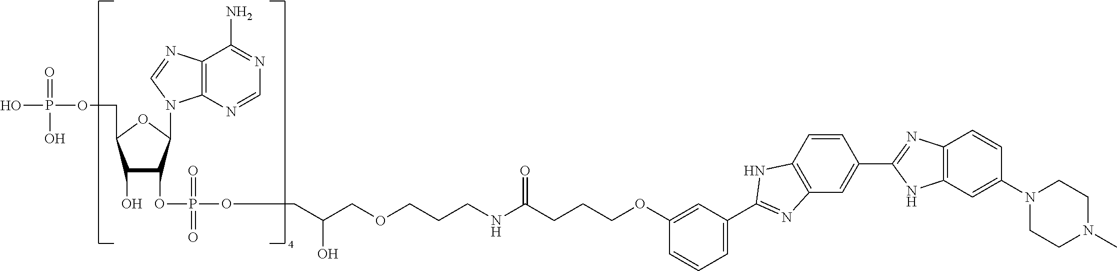 |

### Definitions

As used herein, the term “nucleobase” refers to the base portion of a nucleoside or nucleotide. In certain embodiments, a nucleobase is a purine (also called purinyl) or pyrimidine (also called pyrimidinyl) base. In certain embodiments, the nucleobase is adeninyl, purinyl, thyminyl, cytosinyl, pyrimidinyl, uracilyl, triazolopyridinyl, imidazolopyridinyl, pyrrolopyrimidinyl, triazolopyrimidinyl, pyrazolopyrimidinyl, guaninyl, adeninyl, hypoxanthinyl, 7-deazaguaninyl, 7-deazaadeninyl, or pyrrolotriazinyl. In certain embodiments, a nucleobase is adenine, guanine, cytosine, or thymine. In certain embodiments, a nucleobase is adenine.

As used herein, the term “alkyl” refers to straight chained and branched saturated hydrocarbon groups containing one to thirty carbon atoms, for example, one to twenty carbon atoms, or one to ten carbon atoms. The term C, means the alkyl group has “n” carbon atoms. For example, C₄ alkyl refers to an alkyl group that has 4 carbon atoms. C₁-C₆ alkyl refers to an alkyl group having a number of carbon atoms encompassing the entire range (e.g., 1 to 6 carbon atoms), as well as all subgroups (e.g., 1-6, 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “alkylene” used herein refers to an alkyl group having two points of attachment. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms. An “optionally substituted” moiety may or may not have the recited substituent. For example, C₂₋₆alkylene optionally substituted with one or two OH comprises C₂, C₃, C₄, C₅, and C₆alkylene groups having one or two hydrogen radicals replaced with OH.

As used herein, the term “linker moiety” refers to a straight or branched chain group comprising saturated hydrocarbon groups containing five to one hundred fifty carbon atoms, for example, five to one hundred, five to ninety, ten to eighty, ten to seventy, ten to sixty, ten to fifty, ten to forty carbon atoms, ten to thirty carbon atoms, ten to twenty carbon atoms, or five to fifty carbon atoms, and optionally interrupted with one or more (e.g., 1-20, 1-15, 1-10, 1-5, 1, 2, 3, 4, 5, 6, 7, 8, 9, or 10) heteroatoms (e.g., selected from O, N, S, P, Se, and B, or selected from N, O, and S). Unless otherwise indicated, the chain may be optionally substituted. Substituents can include but are not limited to alkyl, cycloalkyl, alkenyl, cycloalkenyl, alkynyl, heterocycloalkyl, aryl, heteroaryl, hydroxyl, oxo, alkoxy, heteroalkoxy, ester, thioester, carboxy, cyano, nitro, amino, amido, azido, acetamide, and halo (e.g., fluoro, chloro, bromo, or iodo). Linker moieties can be polymer chains, but are not required to be polymeric. Nonlimiting examples of linker moieties include polyalkylene chains (such as polyethylene or polypropylene chains), polyalkylene glycol chains (such as polyethylene glycol and polypropylene glycol), polyamide chains (such as polypeptide chains), and the like. The linker moiety can be attached to the rest of the compound via an amide functional group, an ester functional group, a thiol functional group, an ether functional group, a carbamate functional group, a carbonate functional group, a urea functional group, an alkene functional group, an alkyne functional group, or a heteroaryl ring (e.g., as formed via a Click chemistry reaction between an alkyne and an azide).

As used herein, the term “RNA-targeting group” includes moieties which selectively bind to RNA. RNA-targeting groups can be selective for a particular RNA sequence. RNA-targeting groups can bind to RNA in either a covalent or non-covalent fashion. Non-limiting examples of RNA-targeting groups include small molecules, e.g. targapremir or targaprimir.

As used herein, the term “therapeutically effective amount” means an amount of a compound or combination of therapeutically active compounds (e.g., an mRNA binding compound) that ameliorates, attenuates or eliminates one or more symptoms of a particular disease or condition (e.g., cancer), or prevents or delays the onset of one of more symptoms of a particular disease or condition.

As used herein, the term “patient” means animals, such as dogs, cats, cows, horses, and sheep (e.g., non-human animals) and humans. Particular patients are mammals (e.g., humans). The term patient includes males and females.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

The compounds disclosed herein can be as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds disclosed herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. Also envisioned is the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

### Synthesis of Compounds of the Disclosure

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. For example, the compounds disclosed herein can be synthesized by solid phase synthesis techniques including those described in Merrifield, J. Am. Chem. Soc. 1963; 85:2149; Davis et al., Biochem. Intl. 1985; 10:394-414; Larsen et al., J. Am. Chem. Soc. 1993; 115:6247; Smith et al., J. Peptide Protein Res. 1994; 44: 183; O'Donnell et al., J. Am. Chem. Soc. 1996; 118:6070; Stewart and Young, Solid Phase Peptide Synthesis, Freeman (1969); Finn et al., The Proteins, 3rd ed., vol. 2, pp. 105-253 (1976); and Erickson et al., The Proteins, 3rd ed., vol. 2, pp. 257-527 (1976). The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

Additional synthetic procedures for preparing the compounds disclosed herein can be found in the Examples section.

### Pharmaceutical Formulations, Dosing, and Routes of Administration

Further provided are pharmaceutical formulations comprising a compound as described herein (e.g., compounds of Formula I, Formula Ia, Table A, and pharmaceutically acceptable salts thereof) and a pharmaceutically acceptable excipient.

The compounds described herein can be administered to a subject in a therapeutically effective amount (e.g., in an amount sufficient to prevent or relieve the symptoms of cancer). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, e.g., from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain and may last one day to several months.

Suitable methods of administering a physiologically-acceptable composition, such as a pharmaceutical composition comprising the compounds disclosed herein (e.g., compounds of Formula I, Formula Ia, Table A, or pharmaceutically acceptable salts thereof), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by chemico-physical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods disclosed herein may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, Pa., pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

Disclosed herein are methods of cleaving a nucleic acid comprising contacting the nucleic acid with an effective amount of the compounds or salts disclosed herein. In some cases, the nucleic acid is an RNA.

In some cases, the nucleic acid is an miR-96 precursor hairpin RNA. In some cases, the compound or salt is a compound or salt of formula Ia:

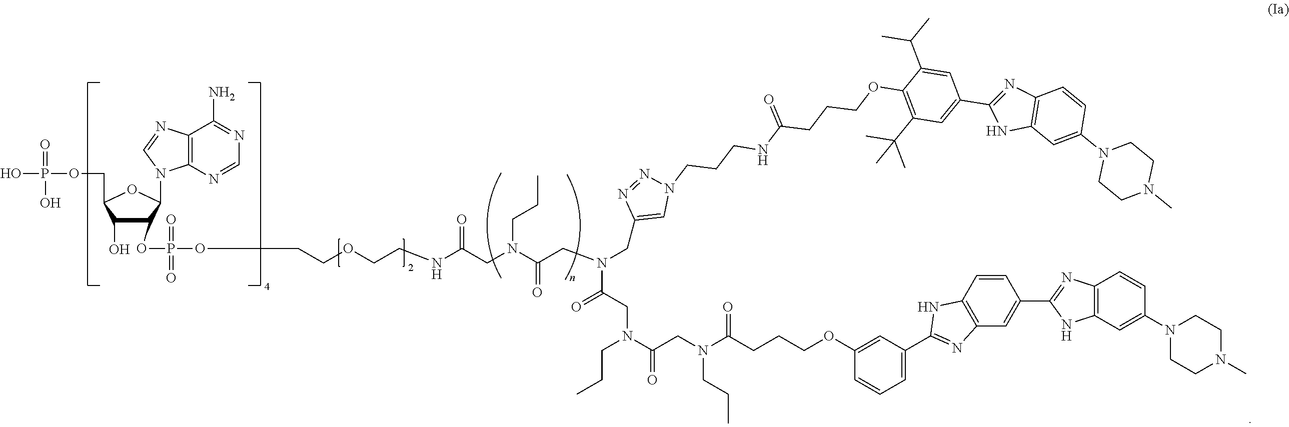

In some cases, the nucleic acid is pre-miR-210 precursor hairpin RNA. In some cases, the compound or salt is a compound of formula I, wherein L is

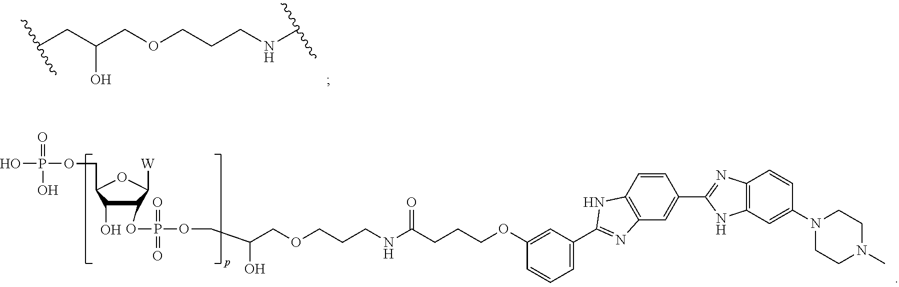

In some cases, contacting occurs inside a cell. In some cases, the cell is a cancer cell. In some cases, the cancer cell is a breast cancer cell.

Also provided are methods of treating a disease or disorder comprising administering to a patient in need thereof a therapeutically effective amount of the compound or salt disclosed herein. In some cases, the disease or disorder is cancer. In some cases, the cancer is breast cancer. In some cases, the breast cancer is triple negative breast cancer.

In some cases, administering the compound or salt de-represses pro-apoptotic FOXO1 transcription factor in a cell. In some cases, de-repression of pro-apoptotic FOXO1 transcription factor triggers apoptosis in a breast cancer cell. In some cases, the therapeutically effective amount of the compound or salt triggers apoptosis in a breast cancer cell. In some cases, the therapeutically effective amount of the compound or salt does not trigger apoptosis in a healthy breast cell. In some cases, the therapeutically effective amount of the compound or salt does not bind to DNA, or binds to DNA at least 5 fold less than to RNA. In some cases, the therapeutically effective amount of the compound or salt does not bind to DNA. In some cases, the therapeutically effective amount of the compound or salt binds to DNA at least 5-fold less than to RNA. In some cases, the compound binds to DNA at least 6-fold, at least 7-fold, at least 8-fold, at least 9-fold, or at least 10-fold less than to RNA, or up to fifty times less than to RNA.

Also disclosed herein are methods of cleaving RNA comprising contacting the RNA with a compound, or pharmaceutically acceptable salt thereof, having a structure of A₂₋₄-linker-Ht, wherein A is adenosine, linker comprises 5 to 150 carbon atoms optionally interrupted with 1 to 20 heteroatoms individually selected from N, O and S, and Ht is an RNA-targeting group. In some cases, Ht comprises

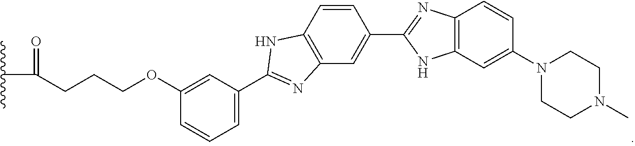 [SMILES: *C(=O)CCCOc1cccc(-c2nc3cc(-c4nc5ccc(N6CCN(C)CC6)cc5[nH]4)ccc3[nH]2)c1]

In some cases, the compound or salt comprises A₄-linker-Ht.

The ENCODE project showed that only 1-2% of the genome encodes for protein, yet 70-80% is transcribed into RNA. Not surprisingly, non-coding RNAs play myriad roles in cellular biology including regulating protein production and gene expression and functioning in immune response. As key regulators of cellular function, RNA production and destruction are tightly controlled.

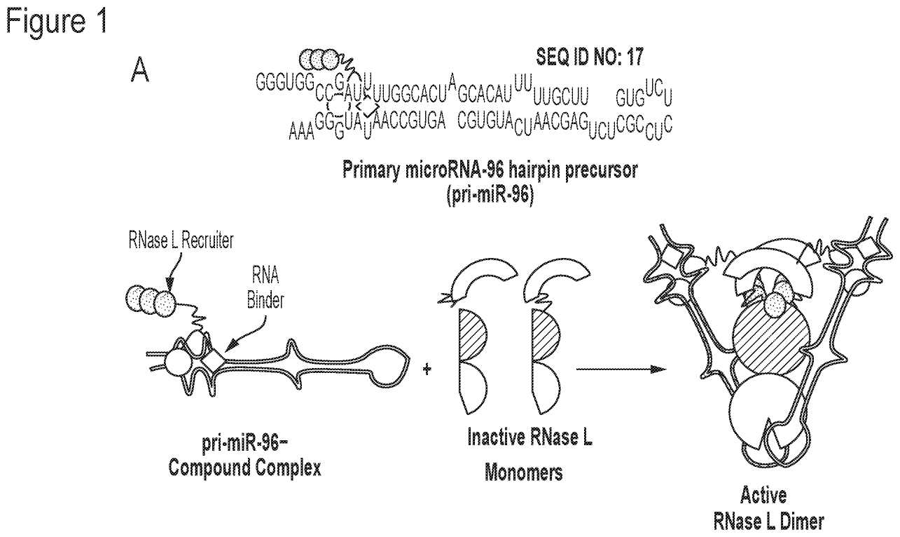

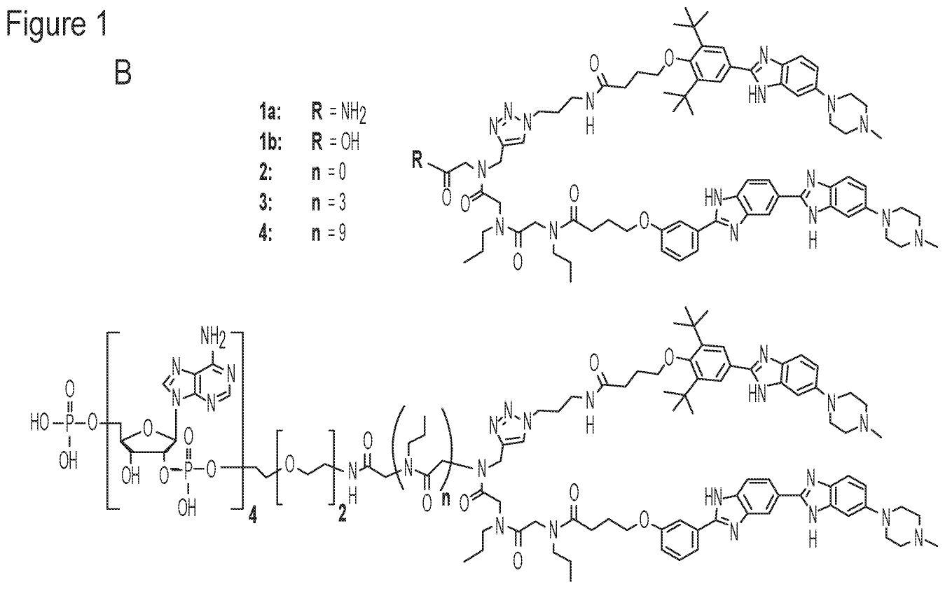

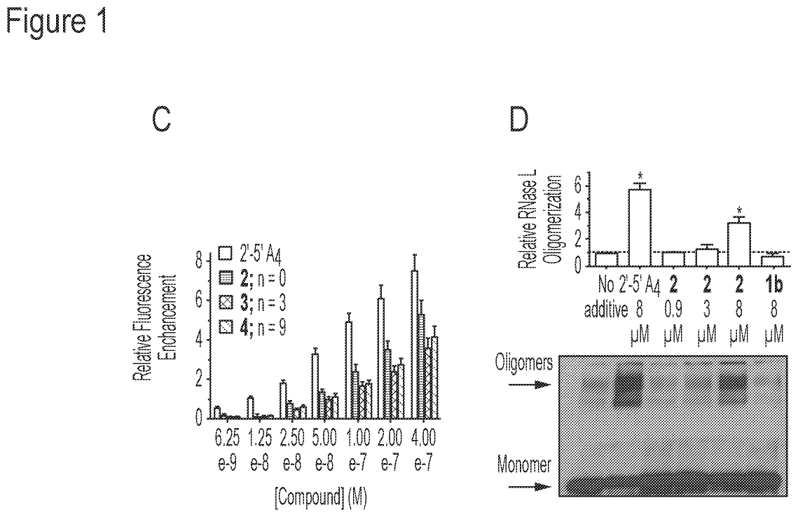

Proteolysis targeting chimeras (PROTACs) are a proven approach for targeted protein degradation by using small molecules. A potential approach to mediate RNA decay is to exploit ribonucleases (RNases) that naturally regulate RNA lifetime and recruit them to specific transcripts via a small molecule, or Ribonuclease targeting chimeras (RIBOTACs). RNase L, an integral part of the antiviral immune response, is present in minute quantities in all cells as an inactive monomer. Upon activation of the immune system, RNase L is upregulated and 2′-5′ oligoadenylate [2′-5′poly(A)] is synthesized; binding of 2′-5′poly(A) dimerizes and activates RNase L (FIG. 1A). Due to the ubiquitous nature of this system, assembling active RNase L onto a specific RNA target to cleave it, akin to antisense, represents a novel strategy for modulating RNA and associated activity in vivo.

We show that a small molecule can recruit a nuclease to a specific transcript, triggering its destruction. A small molecule that selectively binds the oncogenic miR-96 hairpin precursor was appended with a short 2′-5′ poly(A) oligonucleotide. The conjugate locally activated endogenous, latent ribonuclease (RNase L), which selectively cleaved the miR-96 precursor in cancer cells. Importantly, the compound demonstrates catalytic cleavage in cells. Silencing miR-96 de-repressed pro-apoptotic FOXO1 transcription factor, triggering apoptosis in breast cancer, but not healthy breast, cells. These results demonstrate that small molecules can be programmed to selectively cleave RNA and has broad implications.

Computational studies have enabled the design of a small molecule, Targapremir-210 (TGP-210), that targets the microRNA-210 hairpin precursor. MicroRNAs are initially synthesized as primary transcripts (pri-miRNAs) in the nucleus and are cleaved by the nuclease Drosha to generate a precursor microRNA hairpin (pre-miRNA) that is translocated to the cytoplasm where the cytoplasmic nuclease Dicer cleaves the RNA to liberate the mature microRNA (miRNA) (Bartel, 2004). These miRNA targets are dysregulated in a variety of disease settings. For example, miR-210 is upregulated in cells that are hypoxic, or are in a low oxygen environment. When cancer cells undergo hypoxia, cells begin to exhibit behavior associated with extracellular matrix remodeling and increased migratory and metastatic properties. In humans, metastatic breast cancer can be detected via a liquid biopsy via miR-210.

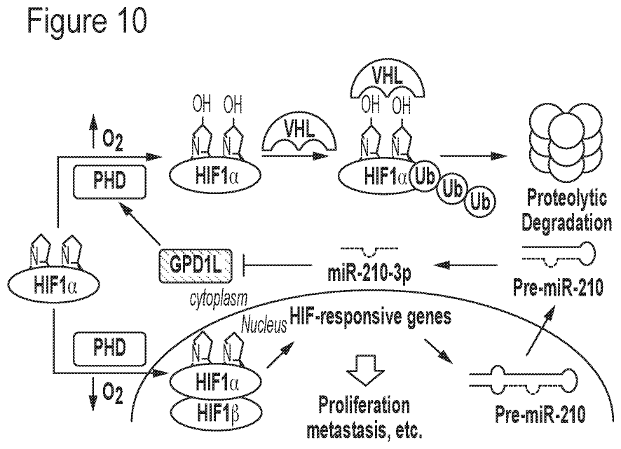

In hypoxic cancers, miR-210 functions by targeting the glycerol-3-phosphate dehydrogenase 1-like (GPD1L) mRNA to repress its translation. In a normoxic environment, the GPD1L protein binds to prolyl hydroxylase (PHD) to promote hyperhydroxylation of hypoxia inducible factor 1-alpha (HIF1α), thus mediating the polyubiquitination and subsequent degradation of this protein by the proteasome (FIG. 10). At low oxygen concentrations, such as in solid breast cancer tumors, miR-210 represses GPD1L mRNA that in turn, decreases PHD activity, stabilizing cytoplasmic HIF1β levels, allowing for its dimerization with hypoxia inducible factor 1-beta (HIF1β) in the nucleus, to form the active HIF1 transcription factor to turn on hypoxia-associated genes (FIG. 10). As miR-210 is among the genes upregulated by HIF1, overexpressed miR-210 triggers a positive feedback loop to drive further miR-210 expression.

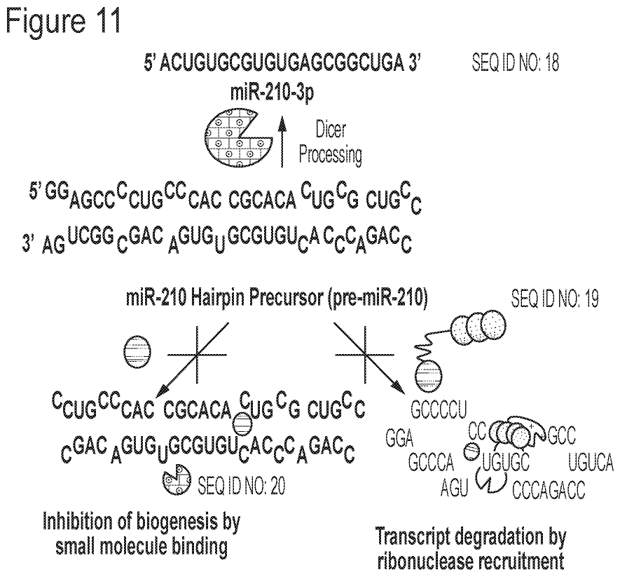

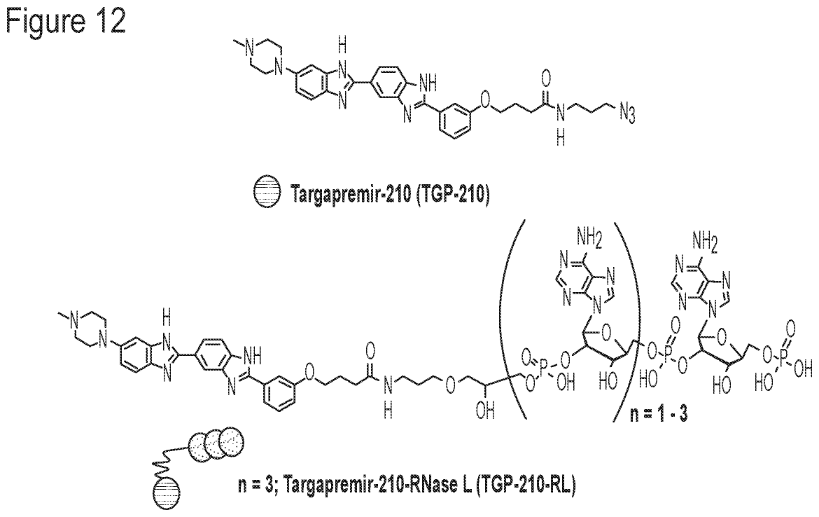

The small molecule TGP-210 targets the Dicer site in pre-miR-210 and inhibits its Dicer processing, thus decreasing the biogenesis of mature miR-210-3p (FIG. 11). This lead compound disrupted downstream hypoxic processes by enhancing GPD1L production to cause HIF1α dysregulation, resulting in apoptosis of only hypoxic cells. Apoptosis caused by the TGP-210 compound also inhibited tumor growth in a triple negative breast cancer mouse model and is a lead targeted therapeutic. In this study, we apply targeted RNA degradation approaches to improve the activity of the TGP-210 small molecule (FIGS. 11 and 12).

An RNA targeting compound that targets the Drosha endonuclease processing site of microRNA (miR)-96 was identified termed Targaprimir-96 (1a). This molecule selectively inhibited biogenesis of miR-96, de-repressed pro-apoptotic transcription factor FOXO1 (a target of miR-96), and triggered apoptosis selectively in triple negative breast cancer cells (MDA-MB-231). To study if RNase L could be effectively recruited to cleave the primary transcript of miR-96 (pri-miR-96), the distance between the RNA targeting moiety of 1a and a short 2′-5′A₄ oligonucleotide, which functions as an RNase L recruiter was varied (2-4, FIG. 1B). In vitro fluorescence-based and gel cleavage experiments showed that compounds triggered cleavage of pri-miR-96 and that the shortest spacer (2) was most effective (FIG. 1C). Importantly, addition of the 2′-5′A₄ component of 2 did not significantly affect its avidity for pri-miR-96's Drosha site, as compared to the parent compound 1a⁹ (K_{d}=20 nM), and no saturable binding was observed to a fully base paired RNA.

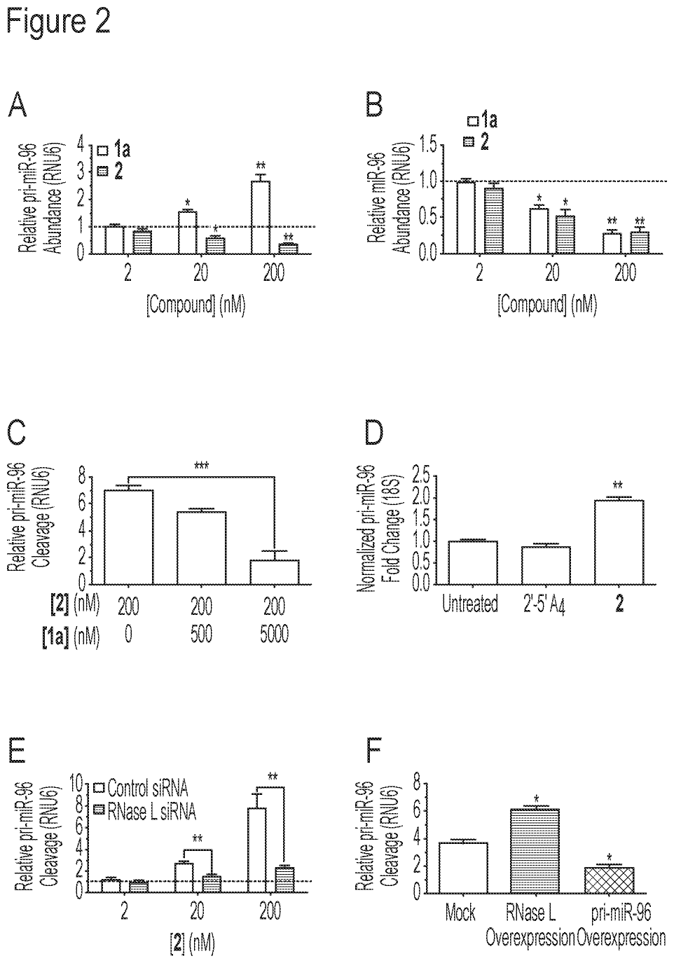

To ensure that the effect of 2 on pri-miR-96 levels was due to recruitment of RNase L, a series of experiments were completed. The addition of 2′-5′A₄ alone, either directly to cell culture or by forced cellular uptake (transfection), had no effect on pri-miR-96 levels. These results suggest that cleavage of pri-miR-96 by 2 is due to specific recruitment of RNase L to pri-miR-96 in cells, as opposed to general stimulation of the RNase L pathway. Further, addition of 2 had no effect on RNase L mRNA levels. Cells were co-treated with a constant concentration of 2 and increasing concentrations of compound 1a, which targets the same site on pri-miR-96 but does not recruit RNase L. The effect of co-treatment on pri-miR-96 levels was then measured. As expected, addition of 1a decreased pri-miR-96 cleavage levels in a dose dependent fashion (FIG. 2C), indicating that 2 directs RNase L to pri-miR-96 and induces its cleavage and can be competed off with a non-recruiting compound. To validate that an RNase L-2-pri-miR-96 ternary complex forms, RNase L was immunoprecipitated from cells treated with 2 or 2′-5′A₄. A ˜2-fold enhancement of the pri-miR-96 transcript was observed from cells treated with 2 as compared to cells treated 2′-5′A₄ (both normalized relative to background pull-down of β-actin; FIG. 2D). Indeed, 2 is selective for formation of the ternary complex with pri-miR-96 as pri-miR-210 is not pulled down (RNAs bound to RNase L activated by 2′-5′A₄ or 2 show no enrichment of the pri-miR-210 transcript, as measured by RT-qPCR. The pri-miR-210 transcript was chosen because a fragment of 2 contains an-RNA binding module that can bind to the miR-210 hairpin precursor.)

It was then investigated whether selective recruitment of RNase L by 2 to pri-miR-96 was operational in cells. As oligonucleotides are generally not cell permeable, the cellular permeability of 2 was measured by flow cytometry. Although conjugation of 2′-5′A₄ reduced permeability of 2 by 50% as compared to 1a it still entered cells unaided and in significant amounts. While distribution of RNase L is mostly cytosolic, nuclear fractions of RNase L have also been observed.

Therefore, if 2 recruits RNase L to pri-miR-96 in the nucleus, a reduction of both pri-miR-96 and mature miR-96 levels is expected. Indeed, both levels were reduced in MDA-MB-231 cells (FIG. 2A-B), which overexpress miR-96. These results were recapitulated in other cancer cell lines that express miR-96, suggesting broad applicability. In contrast, addition of 1a increased the amount of the pri-miR-96 and reduced levels of mature miR-96 (FIG. 2A-B), as expected, as the compound inhibits cleavage by Drosha endonuclease. Importantly, these results indicate that cleavage of miRNAs by enzymes other than their canonical processing enzymes directs them to an RNA decay pathway. That is, cleavage by RNase L degrades pri-miRNA, resulting in decreased mature miRNA, rather than acting as an alternative Drosha endonuclease, leading to an increase in mature miRNA.

Recruitment of RNase L by 2 with 2′-5′A₄ in vitro was assessed. Compared to the natural substrate, 2′-5′ A₄, compound 2 assembled RNase L into its active oligomeric species at 50% lower levels at the same concentration (FIG. 1D). Next, the capacity of these compounds to cleave pri-miR-96 was studied in vitro. Consistent with the preferred cleavage sites of RNase L, one major cleavage site was observed when 2′-5′A₄ alone was added (U12, the Drosha site. RNase L alone caused no significant cleavage.

Interestingly, upon addition of the parent compound, the amount of cleavage by RNase L did not change; rather, the major cleavage site became U35, indicating that the parent compound blocked cleavage by binding the Drosha site as designed and had no inhibitory activity on RNase L. The analogous experiments with 2 revealed that it effectively activated RNase L and recruited the enzyme to pri-miR-96 to U12, the major cleavage site observed for 2′-5′A₄. Further, cleavage at U12 was inhibited by addition of the parent compound (1a) as expected. In contrast to 2′-5′A₄, the small molecule, which drives affinity for the Drosha site alone, did not allow 2 to recruit RNase L to other sites, i.e., U35 for 2′-5′A₄, indicating that 2 is selective. This observation was further bolstered by studying cleavage of pri-miR-96 in the presence of increasing concentrations of tRNA. Indeed, addition of tRNA more significantly inhibited cleavage of pri-miR-96 by 2′-5′A₂ than by 2.

Next, RNase L was knocked down by using siRNA, which should reduce 2's activity. In cells treated with siRNA directed at RNase L, but not a scrambled siRNA, 2's activity was reduced to levels observed without treatment, as determined by measuring pri-miR-96 cleavage activity (FIG. 2E). Conversely, forced expression of RNase L enhanced 2's cleavage activity while forced expression of pri-miR-96 decreased cleavage activity (FIG. 2F). These gain- and loss-of-function experiments further support the assertion that 2 cleaves a specific RNA transcript through recruitment of RNase L in cells. Importantly, 2 was exposed to cells and the cellular fraction of 2 was isolated and analyzed. These results show that 2 is stable in cells. Therefore, cleavage of pri-miR-96 occurred through the chimeric properties of 2, rather than through the separate effects of 2′-5′ A₄ and 1a.

It was next determined whether selective recruitment of RNase L by 2 to pri-miR-96 was operational in cells. As oligonucleotides are generally not cell permeable, the cellular permeability of 2 was measured by flow cytometry. Although conjugation of 2′-5′A₄ reduced permeability of 2 by 50% as compared to 1a it still entered cells unaided and in significant amounts. While distribution of RNase L is mostly cytosolic, nuclear fractions of RNase L have also been observed. Therefore, if 2 recruits RNase L to pri-miR-96 in the nucleus, a reduction of both pri-miR-96 and mature miR-96 levels is expected. Indeed, both levels were reduced in MDA-MB-231 cells (FIG. 2A-B), which overexpress miR-96. These results were recapitulated in other cancer cell lines that express miR-96, suggesting broad applicability. In contrast, addition of 1a increased the amount of the pri-miR-96 and reduced levels of mature miR-96 (FIG. 2A-B), as expected, as the compound inhibits cleavage by Drosha endonuclease. Importantly, these results indicate that cleavage of miRNAs by enzymes other than their canonical processing enzymes directs them to an RNA decay pathway. That is, cleavage by RNase L degrades pri-miRNA, resulting in decreased mature miRNA, rather than acting as an alternative Drosha endonuclease, leading to an increase in mature miRNA.

To ensure that the effect of 2 on pri-miR-96 levels was due to recruitment of RNase L, a series of experiments were completed. The addition of 2′-5′A₄ alone, either directly to cell culture or by forced cellular uptake (transfection), had no effect on pri-miR-96 levels. These results suggest that cleavage of pri-miR-96 by 2 is due to specific recruitment of RNase L to pri-miR-96 in cells, as opposed to general stimulation of the RNase L pathway. Further, addition of 2 had no effect on RNase L mRNA levels. Cells were co-treated with a constant concentration of 2 and increasing concentrations of compound 1a, which targets the same site on pri-miR-96 but does not recruit RNase L, The effect of co-treatment on pri-miR-96 levels was then measured. As expected, addition of 1a decreased pri-miR-96 cleavage levels in a dose dependent fashion (FIG. 2C), indicating that 2 directs RNase L to pri-miR-96 and induces its cleavage and can be competed off with a non-recruiting compound. To validate that an RNase L-2-pri-miR-96 ternary complex forms, RNase L was immunoprecipitated from cells treated with 2 or 2′-5′A₄. A ˜2-fold enhancement of the pri-miR-96 transcript was observed from cells treated with 2 as compared to cells treated 2′-5′A₄ (both normalized relative to background pull-down of β-actin; FIG. 2D). Indeed, 2 is selective for formation of the ternary complex with pri-miR-96 as pri-miR-210 is not pulled down.

Next, RNase L was knocked down by using siRNA, which should reduce 2's activity. In cells treated with siRNA directed at RNase L, but not a scrambled siRNA, 2's activity was reduced to levels observed without treatment, as determined by measuring pri-miR-96 cleavage activity (FIG. 2E). Conversely, forced expression of RNase L enhanced 2's cleavage activity while forced expression of pri-miR-96 decreased cleavage activity (FIG. 2F). These gain- and loss-of-function experiments further support the assertion that 2 cleaves a specific RNA transcript through recruitment of RNase L in cells. Importantly, 2 was exposed to cells and the cellular fraction of 2 was isolated and analyzed. These results show that 2 is stable in cells. Therefore, cleavage of pri-miR-96 occurred through the chimeric properties of 2, rather than through the separate effects of 2′-5′ A₄ and 1a.

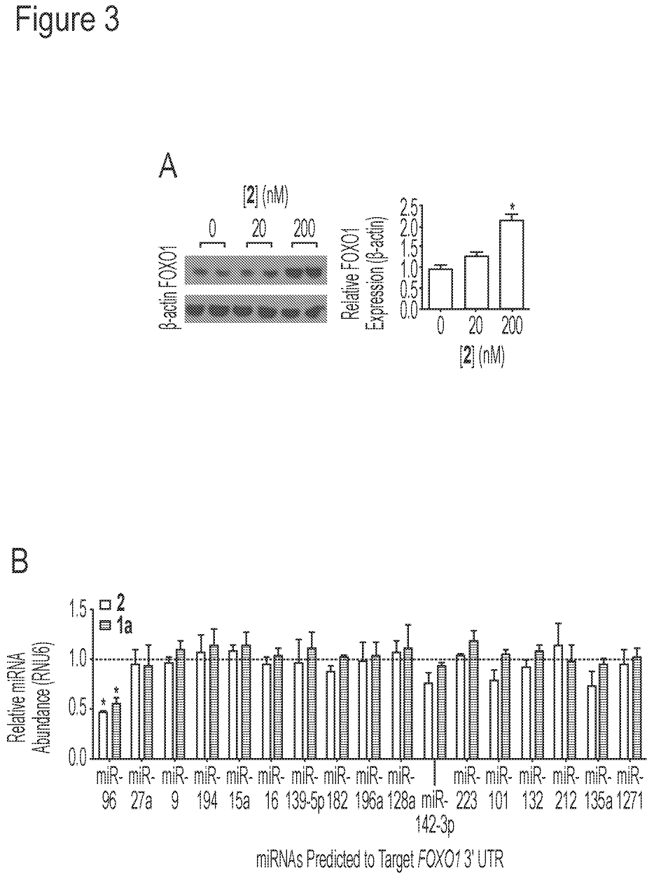

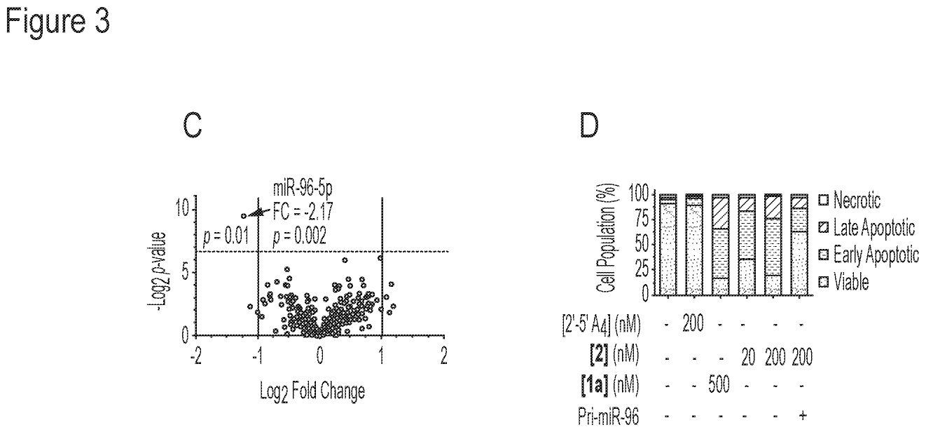

Elevated levels of miR-96 contributes to an invasive phenotype in various cancers due to repression of forkhead box protein O1 (FOXO1), a pro-apoptotic transcription factor required for transcription of pro-apoptotic Bcl-xl proteins. Addition of 2 (200 nM) to MDA-MB-231 cells increased expression of FOXO1 by ˜2-fold while having no effect on a protein not regulated by miR-96 (FIG. 3A). Although FOXO1 mRNA is regulated by miR-182, miR-27a, and miR-96, previous studies have shown that inhibition of miR-96 alone is sufficient to enhance FOXO1 expression. Importantly 2 did not affect levels of miR-27a, miR-182, or other miRNAs predicted to regulate FOXO1 mRNA by TargetScan (FIG. 3B), supporting that the increase in FOXO1 protein expression is mediated through inhibition of miR-96. To further study selectivity, we assessed the effect of 2 on all measurable mature miRNA levels in MDA-MB-231 cells, the most significantly inhibited of which was miR-96 (p<0.01) (FIG. 3C). Since FOXO1 is pro-apoptotic, its de-repression of cellular expression through inhibition of miR-96 should trigger apoptosis. At both 20 and 200 nM, 2 induced significant apoptosis in MDA-MB-231 cells, and 2's ability to trigger apoptosis is ablated upon forced expression of pri-miR-96 (FIG. 3D). Notably, direct treatment or transfection of 2′-5′A₄ alone, at effective concentrations, does not significantly induce apoptosis (FIG. 3D), which is observed upon global activation of the RNase L surveillance system. Additionally, 2 did not induce apoptosis in healthy breast epithelial cells (MCF10a). Thus 2 is a precision chemical probe affecting the biology of cells that express high levels of miR-96. Notably, 2 stimulates apoptosis to the same extent as 1a but at a 2.5-fold lower dose. Given that 2 is taken up at half the amount of 1a, recruitment of RNase L enhances the activity by at least 5-fold.

There is the potential of 2 to catalytically cleave pri-miR-96 in cells and thus this possibility was studied. The absolute levels of cellular pri-miR-96 were measured by RT-qPCR quantification and compared to the amount of 2 in cells. These studies showed that a mole of 2 cleaves, on average, 3.1 moles of pri-miR-96. A turnover of 3.1 agrees well with previous studies of in vitro catalysis of PROTACs.

Thus, cleavage can occur catalytically with targeted recruitment in cells.

In summary, a system is provided herein to endow small molecules with the ability to affect RNA lifetime by recruiting endogenous ribonucleases (RIBOTACs), inducing their cleavage akin to antisense and CRISPR. The ability to custom recruit nucleases is likely to broaden the view of RNA as a viable small molecule target and such parallels can be made to the activities in leveraging PROTACS as chemical probes and lead medicines. Further endeavors will include broadening the nucleases that can be recruited and also to medicinally optimize the recruitment moiety.

### TGP-210-RL Studies

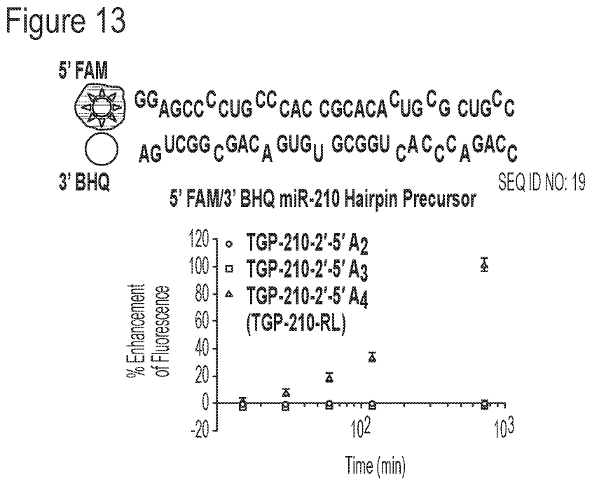

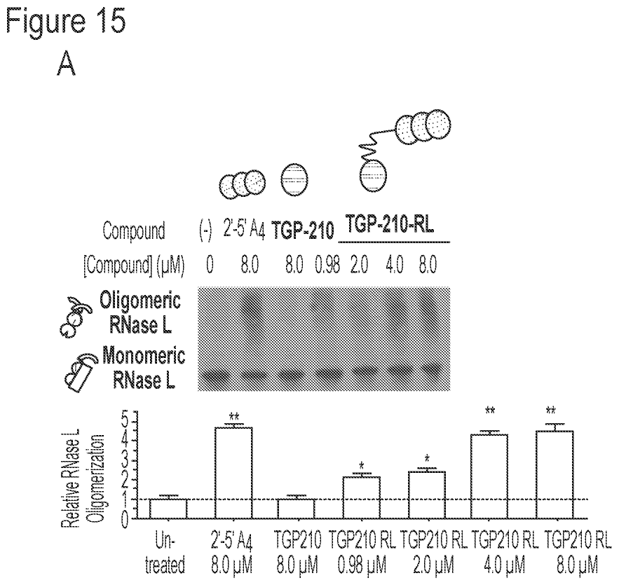

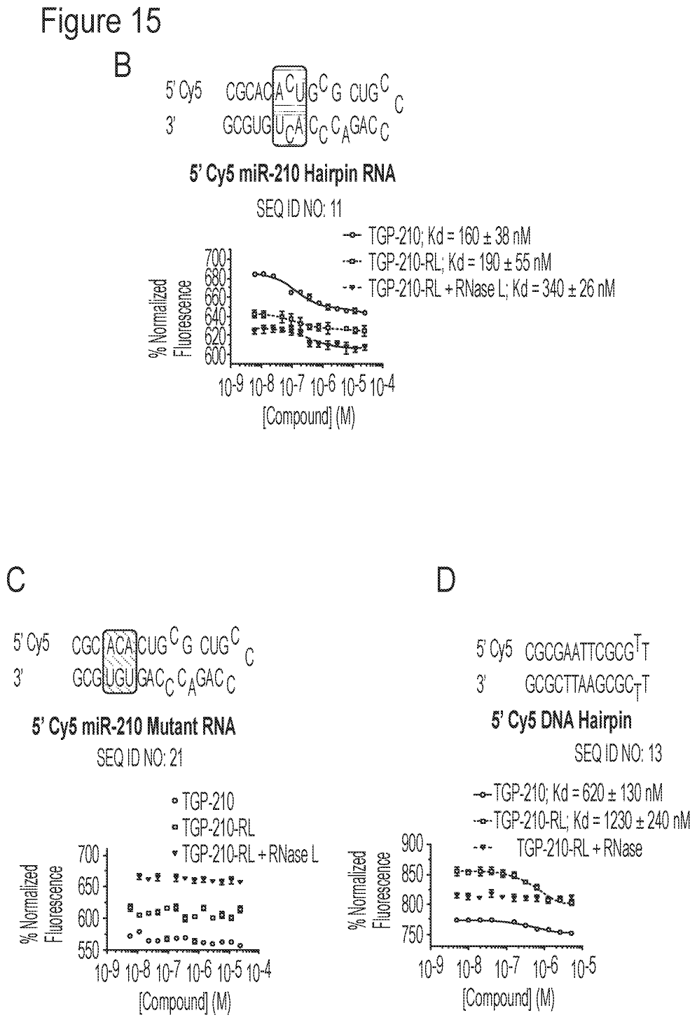

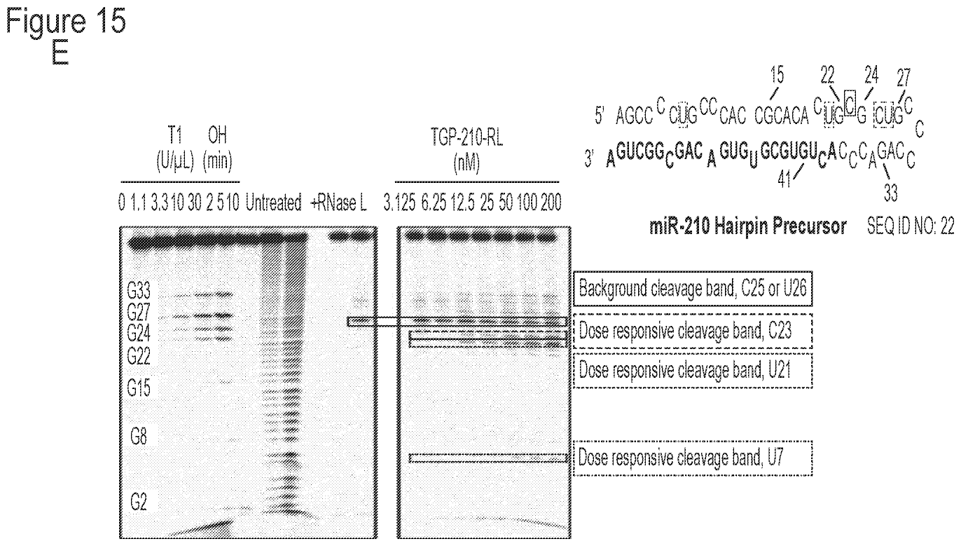

Previously, it has been shown that attachment of short 2′-5′-linked oligoadenylate units (2′-5′ A) to RNA-binding small molecules allows for the local targeted recruitment of latent ribonuclease (RNase L) to cleave RNAs in cells to which the small molecule binds. RNase L is an interferon-inducible endonuclease that, upon activation by 2′-5′ A, cleaves RNA in response to viral infections. Various units of 2′-5′ Aₙ (n=2−4) were attached to TGP-210 and tested the ability of these compounds to cleave pre-miR-210 using a fluorescent in vitro cleavage assay (FIGS. 12 and 13). These studies showed that TGP-210 linked to 2′-5′ A₄ (heretofore named TGP-210-RL) had the most potent cleavage effect (FIG. 13). Very limited cleavage activity was observed for the TGP-210 derivatives appended with the dimer and trimer 2′-5′ oligoadenylates (FIG. 13). Activation of RNase L, which is present in an inactive monomeric form in cells, only occurs upon its oligomerization by binding to 2′-5′ A, thus TGP-210-RL was tested for its ability to activate RNase L. In vitro cross-linking studies of RNase L showed dose-responsive oligomerization of RNase L with TGP-210-RL, but not with TGP-210 treatment (FIG. 15A).

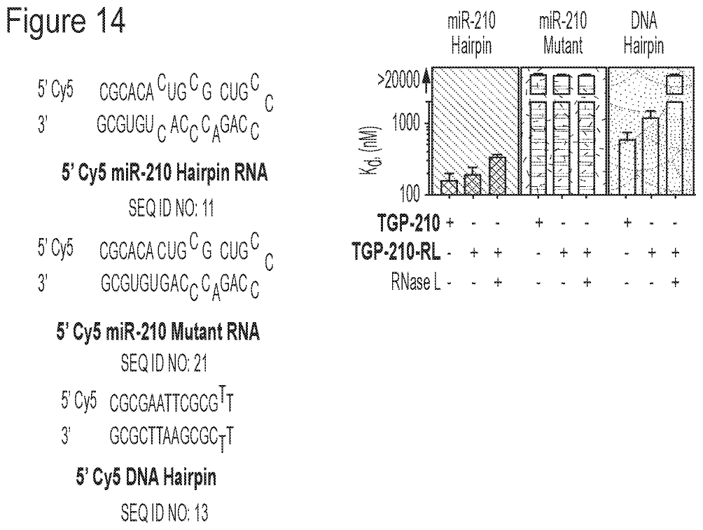

The parent TGP-210 compound is known to bind DNA with a 5-fold selectivity window over the Dicer site in pre-miR-210 (K_{d} to DNA is 620 nM while K_{d} to pre-miR-210 mimic is 160 nM) (FIGS. 14 and 15B-D). To study the binding consequences of adding the 2′-5′ A₄ nuclease recruiting module, binding affinities were measured by microscale thermophoresis (MST) to these targets with TGP-210-RL. In these studies, the affinity for a pre-miR-210 mimic is modestly weaker compared to TGP-210 with a K_{d} of 190 nM (FIGS. 14 and 15B). The binding of TGP-210-RL to DNA increased to a 10-fold window of selectivity, occurring with a K_{d} of 1200 nM (FIG. 14). The TGP-210-RL did not bind to an RNA in which the Dicer site was mutated to a base pair, further demonstrating selective binding (FIGS. 14 and 15C). Since reports from heterobifunctional, PROTACs have shown that ternary complex formation (target:PROTAC:ligase) is important for activity and that PROTACs can have higher selectivity than their respective protein binding modules, the binding affinity of TGP-210-RL for pre-miR-210 and DNA was measured in the presence of RNase L. These studies showed that TGP-210-RL maintained selective binding to RNA with a K_{d} of 340 nM to pre-miR-210, while DNA binding was completely ablated with no measurable binding (FIGS. 14 and 15D). Thus, addition of the recruiter enhanced the binding selectivity of the RNA-targeted small molecule in vitro. Indeed, TGP-210-RL binding and recruitment of RNase L enabled in vitro cleavage of pre-miR-210 as observed by gel (FIG. 15E). The biophysical characteristics of the ternary complex are extremely important to tune for affecting biological activity as has been shown in analyses of targeted protein degraders.

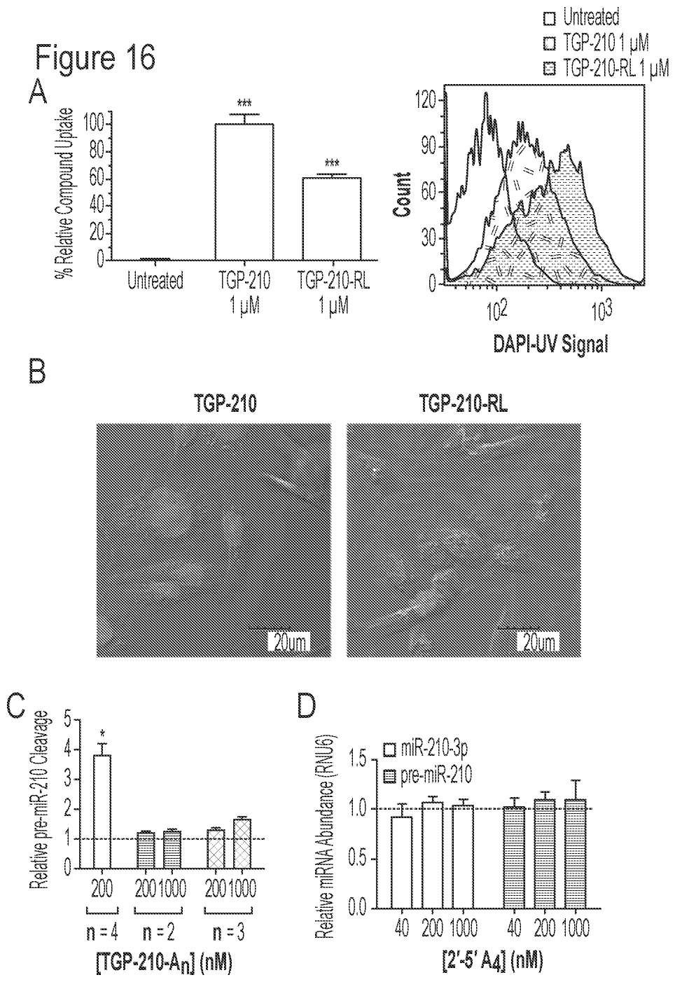

The compound TGP-210-RL was next tested for cellular permeability. The molecule was freely cell permeable and despite having the short oligonucleotide, it entered cells at 60% of the amount relative to the parent compound TGP-210, as measured by flow cytometry (FIG. 16A). Further, confocal microscopy was completed and the intrinsic fluorescence of the parent TGP-210 compound was localized mainly to the nucleus while the signal from TGP-210-RL was localized to the cytoplasm (FIG. 16B). Similar to other short length, cell-permeable modified oligonucleotides, significant cell uptake of the TGP-210-RL small molecule-oligoadenylate conjugate was observed. The DNA off-targets are exclusively nuclear while the RNA, pre-miR-210, is exclusively cytoplasmic. Furthermore, RNase L is predominantly localized to the cytoplasm in confluent cells. Collectively, both the binding affinity and localization experiments suggest that addition of the RNase L recruiting module enhanced the properties of the chimera for targeting pre-miR-210.

To assess the cellular effects of the compounds, TGP-210, TGP-210-RL, and 2′-5 A₄ were tested for affecting miR-210 levels in hypoxic MDA-MB-231 cells. Both TGP-210 and TGP-210-RL decreased the levels of mature miR-210 as expected. An increase in pre-miR-210 levels was observed with TGP-210 treatment, which is expected as the compound inhibits Dicer processing of this RNA in cells. In contrast, TGP-210-RL decreased the levels of both miR-210 and pre-miR-210, and these results are expected if the compound actively cleaves the pre-miR-210 target transcript. Only TGP-210 linked with 4 units of 2′-5′ A caused cleavage in cells, similar to the in vitro results (FIG. 16C). Addition of the 2′-5′ A₄ compound resulted in no significant effect on miR-210 and pre-miR-210 at any concentration tested, showing that cleavage is specific for having the RNA binder and the RNase L recruiter on a single compound, TGP-210-RL (FIG. 16D) as has been previously observed.

To study if the pre-miR-210 cleaving capacity of TGP-210-RL was specific for RNase L, a series of control experiments were completed. First, cells were treated concurrently with a constant concentration of TGP-210-RL and increasing amounts of TGP-210 to bind to the pre-miR-210 target and compete off the nuclease recruiter. In these experiments, as the specific cleaving chimera and the parent compound compete for occupancy of the same binding site in pre-miR-210, addition of TGP-210 caused a decrease in cleavage of pre-miR-210. Second, transfection of RNase L to cells and followed by treatment with TGP-210-RL enhanced the cleavage capacity of the compound, while transfection of a plasmid overexpressing the pre-miR-210 target decreased the cleavage capacity of the compound. These gain- and loss-of-function experiments indicate the RNase L dependence and pre-miR-210 selectivity of the cleavage caused by TGP-210-RL. Third, targeted siRNA ablation of RNase L, but not a control siRNA, decreased the cleavage of pre-miR-210 by TGP-210-RL. These studies demonstrated that TGP-210-RL specifically cleaves pre-miR-210 via the targeted recruitment of RNase L to the target.

To measure if TGP-210-RL physically interacts and forms a ternary complex with RNase L and pre-miR-210 in cells, a co-immunoprecipitation experiment was completed by using an anti-RNase L antibody. In these experiments, immunoprecipitation was completed and the pulled down fraction was subjected to RT-qPCR with primers to detect pre-miR-210. An enrichment of pre-miR-210 in the RNase L-pulled down fraction was observed only when TGP-210-RL was applied in cells, while there was no enrichment in a highly expressed control microRNA hairpin precursor transcript, pre-miR-21. Furthermore, no enrichment of pre-miR-210 was observed with 2′-5′ A₄ treatment, as expected.

The TGP-210-RL compound cleaves pre-miR-210 in a catalytic and substoichiometric fashion. Using the intrinsic fluorescence of TGP-210-RL and quantitative RT-qPCR, the number of molecules of TGP-210-RL and the copies of pre-miR-210 cleaved by TGP-210-RL were measured in cells (Table S1, below). These analyses demonstrate that TGP-210-RL substoichiometrically cleaved 9.7±1.9 molecules of pre-miR-210 per each molecule of TGP-210-RL after treatment for 24 h in cells.

TABLE S1
Measurement of TGP-210-RL catalytic activity after 24 h of treatment.
Data are expressed as mean ± s.d. (n = 6). .

| Treated | TGP-210-RL | Average | Cleaved | |
|---|---|---|---|---|
| [TGP-210-RL] | Detected | pre-miR-210 | pre-miR-210 | |
| (nM) | (pmol) | (pmol) | (pmol) ^{a} | Turnovers ^{b} |
| 0 | — | 300 ± 5.0 | 0 | — |
| 500 | 4.0 ± 0.21 | 260 ± 12 | 39 ± 13 | 9.7 ± 1.9 |

“Cleaved pre-miR-210” is the difference between the Average pre-miR-210 in the untreated and the Average pre-miR-210 in the 500 nM treated samples.
^{b} “Turnovers” is the ratio between “Cleaved pre-miR-210 (pmol)” and “TGP-210-RL Detected (pmol)” in cells and represents catalysis.

Next, the specificity of TGP-210-RL was broadly studied via qPCR profiling to study its effect on all detectable miRNAs in MBD-MB-231 cells. Among over 370 detectible miRNAs, the most significantly inhibited was miR-210, demonstrating that small molecules that bind to pre-miR-210 and locally recruit RNase L are selective. No significant effect of TGP-210-RL on other hypoxia associated miRNAs and pre-miR-210 RNA isoforms, or RNAs with similar structure to pre-miR-210, was observed. As pre-miR-210 ultimately affects GPD1L and thus HIF1α levels, their mRNA transcript levels were measured by RT-qPCR after treatment for 24 h and 48 h. As expected, upon TGP-210-RL treatment, GPD1L mRNA abundance was significantly de-repressed, while HIF1α mRNA was significantly decreased but only after 48 h of treatment. The same result was previously observed with miR-210 inhibition by TGP-210 and miR-210 antisense oligonucleotide treatment.

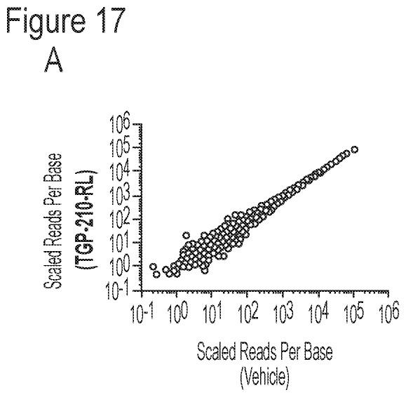

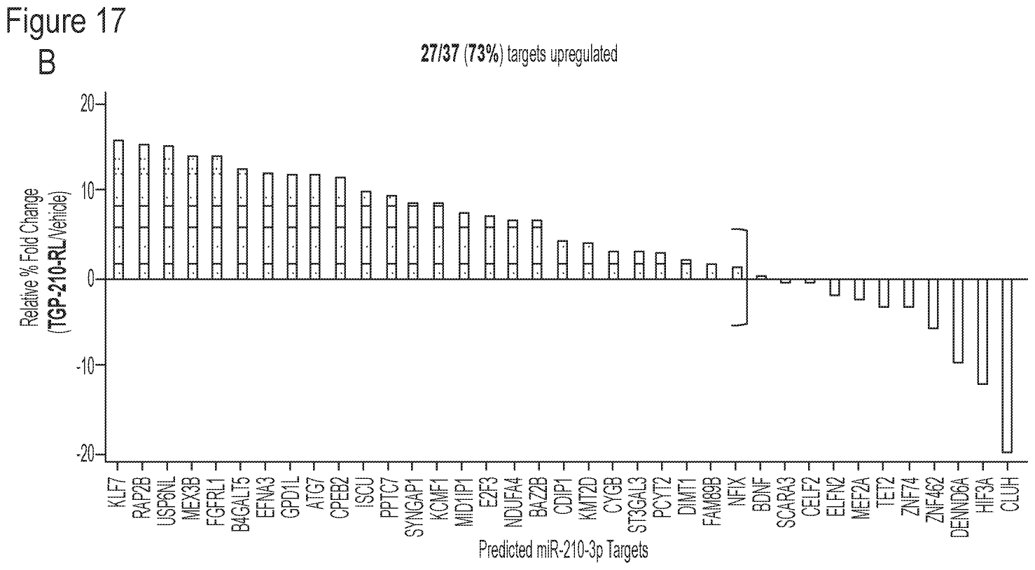

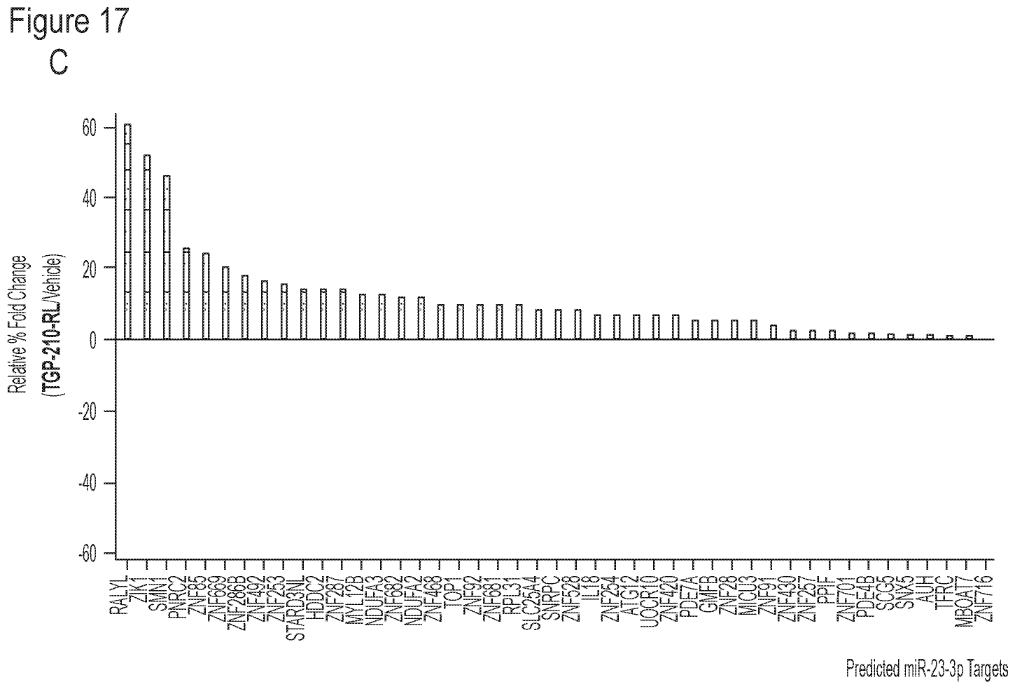

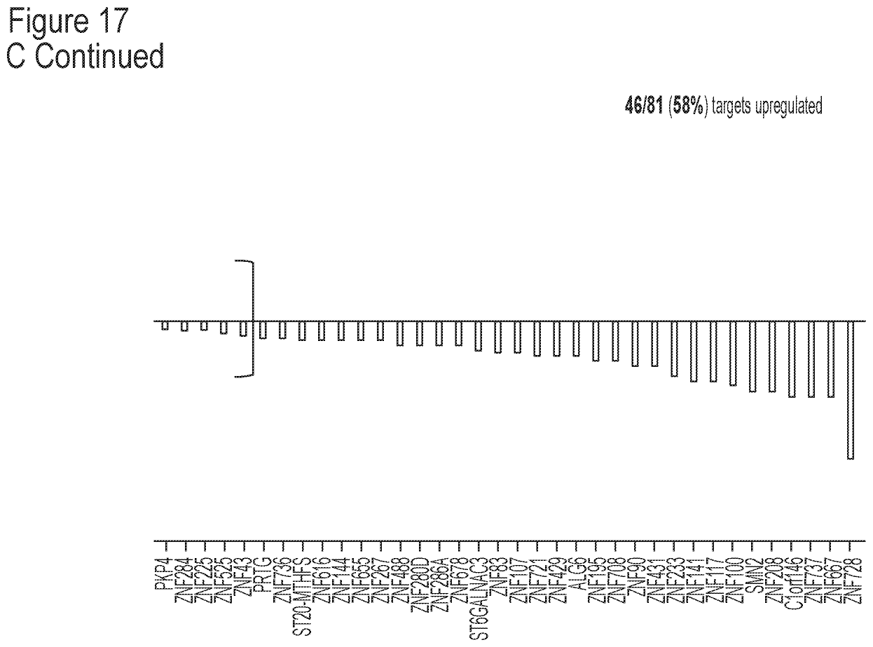

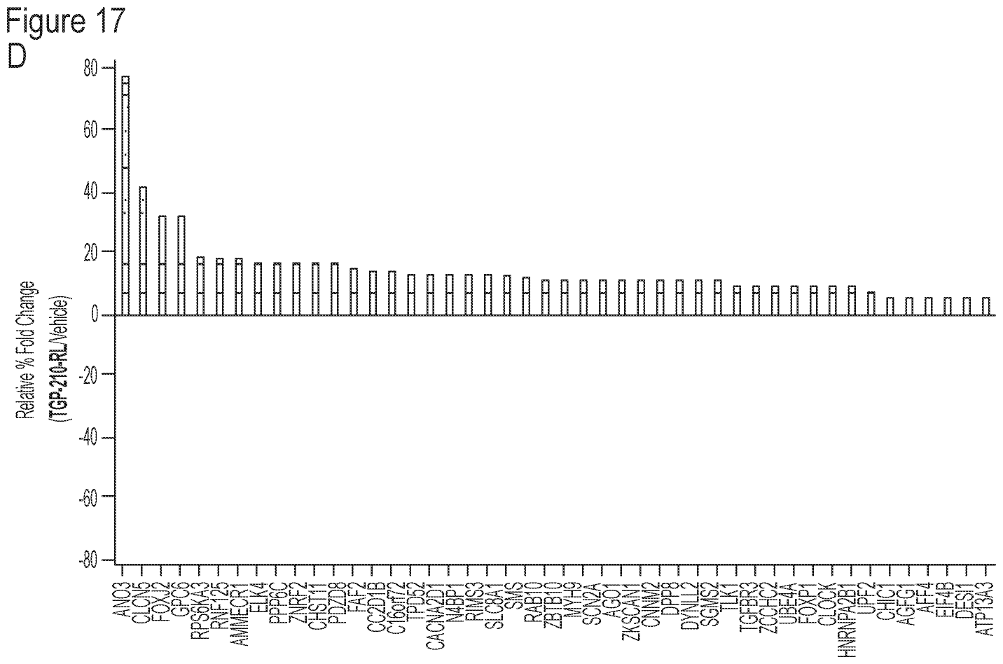

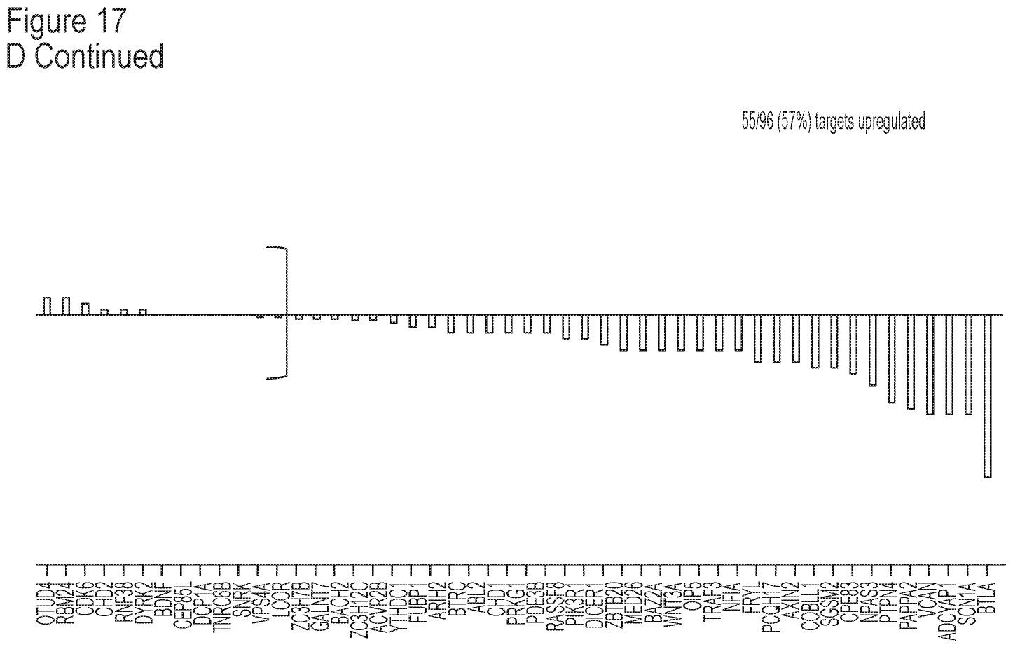

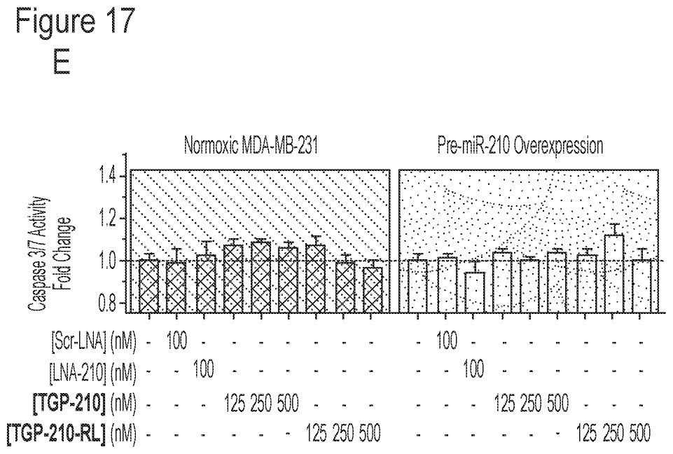

To further study the selectivity of TGP-210-RL, total RNA-Seq was run after 24 h of compound treatment in hypoxia, to avoid measuring indirect effects due to apoptosis. Overall, no major changes to the transcriptome were observed, indicating no significant off-target effects of the compound. The fold changes of predicted miR-210-3p targets were then queried to study on-target effects upon degrading pre-miR-210. Of the miR-210-3p targets, 73% were upregulated in response to compound treatment, relative to the vehicle control, indicating on-target effects of the compound suppressing miR-210-3p. In comparison, the predicted targets of control miRNAs, miR-23-3p and miR-107, which are both highly expressed hypoxia-associated miRNAs, followed a binomial distribution, with no bias in upregulated targets observed, showing 58% and 57% upregulated targets, respectively (FIG. 17C and D). Previous RNA-Seq studies with small molecules targeting miRNA similarly demonstrated limited off-target effects on the transcriptome, while causing a dramatic downstream biological impact and phenotype.

Next, the effect of TGP-210-RL on phenotype was measured. To serve as a positive and negative controls, locked nucleic acid (LNA) oligonucleotides targeting miR-210 (LNA-210) and a scrambled control (Scr-LNA) were studied in addition to TGP-210 and TGP-210-RL. These studies showed that LNA-210 caused apoptotic stimulation while Scr-LNA was inactive, as measured by increased caspase activity. The effect of apoptosis between TGP-210 and TGP-210-RL were similar, but TGP-210-RL displayed greater activity, rivalling that of LNA-210, at 500 nM. Given that TGP-210-RL gets into cells at about 50% of the amount of TGP-210, the nuclease recruitment enhances the activity of the compound by at least 2-fold. One important delineation between a targeted RNA degrader and a binder, however, is that the pre-miRNA is not pervasive in the former case but is in the latter. As a control, these same experiments were completed in a miR-210 overexpressed background, to determine if the compound-induced apoptosis was mediated through miR-210. Indeed, upon miR-210 overexpression, the miR-210-targeting compounds (LNA-210, TGP-210, and TGP-210-RL) no longer stimulated apoptosis, further supporting the hypothesis that apoptosis is due to inhibition of miR-210. Under normoxic conditions, miR-210 is not overexpressed in cells, therefore, the effect of compound on apoptosis in normoxia was measured. No significant increase in apoptosis was observed with compound treatment under normoxic conditions, as expected when cells express lower levels of miR-210 (FIG. 17E). Thus, the small molecule targeted degrader is a lead targeted therapeutic for miR-210.

The compounds described herein (e.g., the compounds of Formula I, Formula Ia, and Table A, and pharmaceutically acceptable salts thereof) can bind nucleic acids. In some embodiments, the compounds bind RNA, e.g., the compounds trigger or inhibit RNA-mediated biological activity, such as gene expression. In various embodiments, the compounds are RNA modulators, e.g., the compounds change, inhibit, or prevent one or more of RNAs biological activities.

Uses of the compounds disclosed herein in the preparation of a medicament for treating diseases and disorders, e.g., cancer, also are provided herein.

The disclosure herein will be understood more readily by reference to the following examples, below.

### EXAMPLES

High throughput time-resolved fluorescence resonance energy transfer (TR-FRET) screening.

### General Synthetic Methods

Chemicals. Chemicals were procured from the following commercial sources: 2-chlorotritylchloride resin (loading=1.1 meq/g), N,N′-diisopropylcarbodiimide (DIC), and Ethyl cyano(hydroxyimino)acetate (Oxyma) from Chem-Impex Int'l Inc.; 1-hydroxy-7-azabenzotriazole (HOAt) from Advanced Chem Tech; 1-[Bis(dimethylamino)methylene]-1H-1,2,3-triazolo[4,5-b]pyridinium 3-oxid hexafluorophosphate (HATU) from Oakwood Chemical; 1-propylamine from Alfa Aesar; propargylamine, trifluoroacetic acid (TFA), N,N-diisopropylethyl amine (DIEA) and 2-bromoacetic acid from Sigma Aldrich; oligonucleotide 5 (lithium salt) from ChemGenes with HPLC purification; and N,N-dimethylformamide (DMF, anhydrous) and dimethyl sulfoxide (DMSO, anhydrous) from EMD and used without further purification.

The stable copper (I) catalyst, 3 Ht-COOH,4 Ht-N3,4 and 1a2 were synthesized as reported previously.

General. Peptide synthesis reactions were monitored by chloranil test. Mass spectrometry was performed with an Applied Biosystems MALDI TOF/TOF Analyzer 4800 Plus using an α-cyano-4-hydroxycinnamic acid matrix (in positive ion mode for 1b, 2a, 3a, 3b, 4a and 4b; in negative ion mode for 2, 3 and 4.)

Compound purification and analysis for 1b, 2a, 3a, 3b, 4a and 4b. Preparative HPLC was performed using a Waters 1525 Binary HPLC pump equipped with a Waters 2487 dual absorbance detector system and a Waters Sunfire C18 OBD 5 μm, 19×150 mm column. Absorbance was monitored at 254 and 220 nm. A linear gradient with a flow rate of 5 mL/min from 0-100% methanol in water with 0.1% TFA over 60 min was used for small molecule purification. Purity was assessed by analytical HPLC using a Waters Symmetry C18 5 μm, 4.6×150 mm column with a flow rate of 1 mL/min and a linear gradient from 0-100% methanol in water with 0.1% TFA over 60 min. Absorbance was monitored at 254 and 220 nm.

Compound purification, concentration and analysis for 2, 3 and 4. Preparative HPLC was performed using a Waters 1525 Binary HPLC pump equipped with a Waters 2487 dual absorbance detector system and a Waters Symmetry C18 5 μm, 4.6×150 mm column. Absorbance was monitored at 254 and 345 nm. A linear gradient with a flow rate of 1 mL/min from 0% to 100% buffer A [50 mM triethylammonium acetate in water/acetonitrile, 50/50 (v/v)] in buffer B (50 mM triethylammonium acetate in water) over 60 min (for 2) or 70 min (for 3 and 4) was used. Collected fractions were evaporated, and the compounds were dissolved in water. The concentration was determined in 1×PBS using a molecular extinction coefficient for 5 (51400 M−1 cm−1 at 260 nm). Purity was assessed by analytical HPLC using a Waters Symmetry C18 5 μm, 4.6×150 mm column. Small molecules were analyzed using a linear gradient with a flow rate of 1 mL/min from 0-100% buffer A in buffer B over 30 min followed by 100% buffer A for 10 min. Absorbance was monitored at 254 and 345 nm.

### Synthetic Experimental Procedures

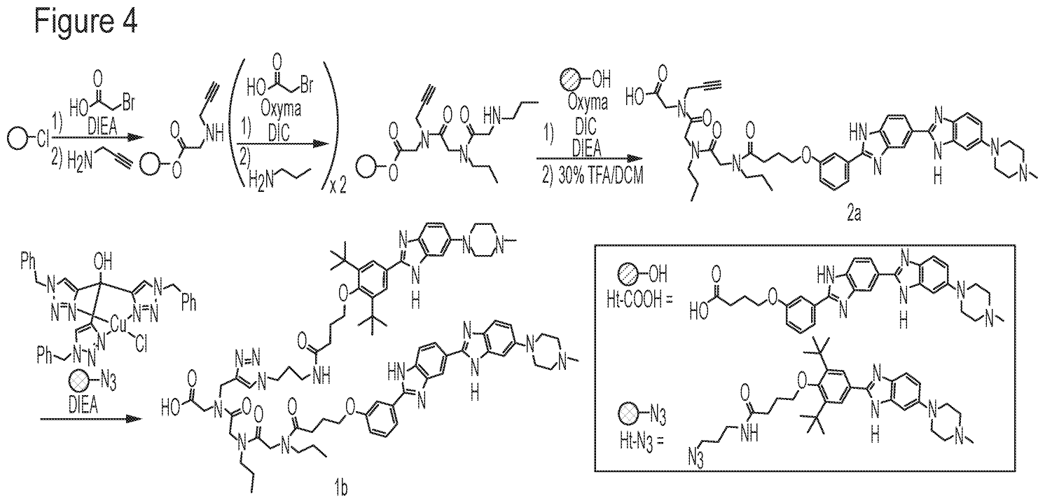

Synthesis of 1b. See FIG. 4. To the suspension of 2-chlorotritylchloride resin (555 mg, 0.61 mmol) in dichloromethane (DCM; 3 mL) was added 4 N HCl in dioxane (1 mL), and the mixture was shaken at room temperature for 30 min. The resin was washed with DMF (3×3 mL) and DCM (3×3 mL). Then to the resin were added 1M bromoacetic acid (3 mL) in DCM and DIEA (519 μL, 3.0 mmol). The mixture was shaken at room temperature for 2 h, and the resin was washed with DCM (3×3 mL) and DMF (3×3 mL). Then, 3 mL of DMF and propargylamine (384 μL, 6.0 mmol) were added. The mixture was shaken at room temperature for 1 h, and the resin was washed with DCM (3×3 mL) and DMF (3×3 mL) followed by addition of 1 M bromoacetic acid (3 mL) in DMF, Oxyma (426 mg, 3.0 mmol) and DIC (462 μL, 3.0 mmol). The mixture was shaken at room temperature for 2 h, and the resin was washed with DCM (3×3 mL) and DMF (3x×3 mL). To the resin were added DMF (3 mL) and propylamine (492 μL, 6.0 mmol). The mixture was shaken at room temperature for 1 h and the resin was washed with DCM (3×3 mL) and DMF (3×3 mL). This cycle was repeated an additional time. Then to the resin was added the mixture of Oxyma (170 mg, 1.2 mmol), DIC (185 μL, 1.2 mmol), DIEA (313 μL, 1.8 mmol) and Ht-COOH (450mg, 0.88 mmol) in DMF (3 mL). The mixture was shaken at room temperature for 2 h and the resin was washed with DCM (3×3 mL) and DMF (3×3 mL). The compound was released from the resin by addition of 30% TFA in DCM (2 mL), and the mixture was shaken at room temperature for 30 min. The mixture was filtered, and the filtrate was evaporated. To the residue was added diethyl ether (Et2O) and the precipitate was collected. The collected powder was purified using reverse phase HPLC as described in the General Synthetic Methods section to afford 2a (275 mg, 56% yield; C44H54N9O6 calculated mass: 804.4197 (M+H)+; found: 804.3752).

To 2a (270 mg, 0.34 mmol) were added stable copper (I) catalyst (12 mg, 0.020 mmol), Ht-N3 (235 mg, 0.40 mmol), DIEA (600 μL, 3.4 mmol) and DMSO (2 mL). The reaction mixture was stirred at 60° C. for 2 h. Then to the reaction mixture was added acetonitrile (10 mL), and precipitate was filtered. To the collected powder was added 10 mL of methanol (MeOH), and the mixture was filtered with filter paper. The filtrate was evaporated, and the residual powder was washed with acetonitrile to afford 1b as a pale yellow powder (91 mg, 0.065 mmol, 22% yield). A portion of 1b was purified using reverse phase HPLC as described in the General Synthetic Methods section. C₇₇H₁₀₂N₁₇O₈ calculated mass: 1392.8089 (M+H)+; found: 1392.7115.

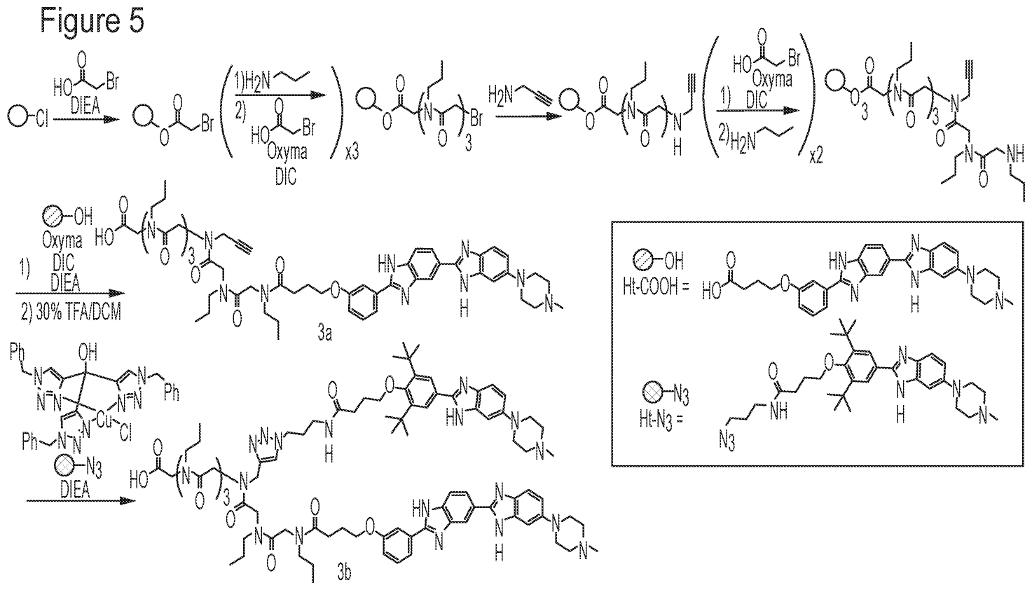

Synthesis of 3b. See FIG. 5. To the suspension of 2-chlorotritylchloride resin (200 mg, 0.22 mmol) in DCM (0.9 mL) was added 4 N HCl in dioxane (0.3 mL) and the mixture was shaken at room temperature for 30 min. The resin was washed with DMF (3×3 mL) and DCM (3×3 mL). Then to the resin were added 1 M bromoacetic acid (1.1 mL) in DCM and DIEA (190 μL, 1.1 mmol). The mixture was shaken at room temperature for 2 h and the resin was washed with DCM (3×3 mL) and DMF (3×3 mL). Next, 1 mL of DMF and propylamine (180 μL, 2.2 mmol) were added, and the mixture was shaken at room temperature for 1 h. After washing the resin with DCM (3×3 mL) and DMF (3×3 mL), 1 M bromoacetic acid (1.1 mL) in DMF, Oxyma (156 mg, 1.1 mmol) and DIC (169 μL, 1.1 mmol) were added to the resin. The mixture was shaken at room temperature for 2 h and the resin was washed with DCM (3×3 mL) and DMF (3×3 mL). This cycle was repeated two additional times. Then to the resin were added 1.1 mL of DMF and propargylamine (141 μL, 2.2 mmol). The mixture was shaken at room temperature for 1 h and the resin was washed with DCM (3×3 mL) and DMF (3×3 mL). After that, to the resin were added 1 M bromoacetic acid (1.1 mL) in DMF, Oxyma (156 mg, 1.1 mmol) and DIC (169 μL, 1.1 mmol). The mixture was shaken at room temperature for 2 h, and the resin was washed with DCM (3×3 mL) and DMF (3×3 mL). After washing, 1.1 mL of DMF and propylamine (180 μL, 2.2 mmol) were added and the mixture was shaken at room temperature for 1 h followed by additional washing (DCM, 3×3 mL and DMF, 3×3 mL). This cycle was repeated an additional time. Then to the resin was added a mixture of Oxyma (85 mg, 0.6 mmol), DIC (93 μL, 0.6 mmol), DIEA (156 μL, 0.9 mmol) and Ht-COOH (225 mg, 0.45 mmol) in DMF (1.1 mL). The mixture was shaken at room temperature for 2 h and the resin was washed with DCM (3×3 mL) and DMF (3×3 mL). Then to the resin was added 30% TFA in DCM (2 mL), and the mixture was shaken at room temperature for 30 min. The mixture was filtered, and the filtrate was evaporated. To the residue was added Et₂O and the precipitate was collected. The collected powder was purified using reverse phase HPLC as described in the General Synthetic Methods section to afford 3a (88 mg; 36% yield; C₅₉H₈₁N₁₂O₉ calculated mass: 1101.6249 (M+H)+; found: 1101.4913.

To 3a (80 mg, 0.072 mmol) were added stable copper (I) catalyst (6 mg, 0.010 mmol), Ht-N₃ (51 mg, 0.087 mmol), DIEA (125 μL, 0.72 mmol) and DMSO (1 mL). The reaction mixture was stirred at 60° C. for 6 h. Then to the reaction mixture was added acetonitrile (10 mL), and the precipitate was filtered. To the collected powder was added MeOH, and the mixture was filtered with filter paper. The filtrate was evaporated, and the residual powder was washed with acetonitrile to afford 3b as a yellow powder (37 mg, 0.021 mmol, 30% yield). A portion of 3b was purified using reverse phase HPLC as described in the General Synthetic Methods section. C₉₂H₁₂₉N₂₀O₁₁ calculated mass: 1690.0150 (M+H)+; found 1689.9568.

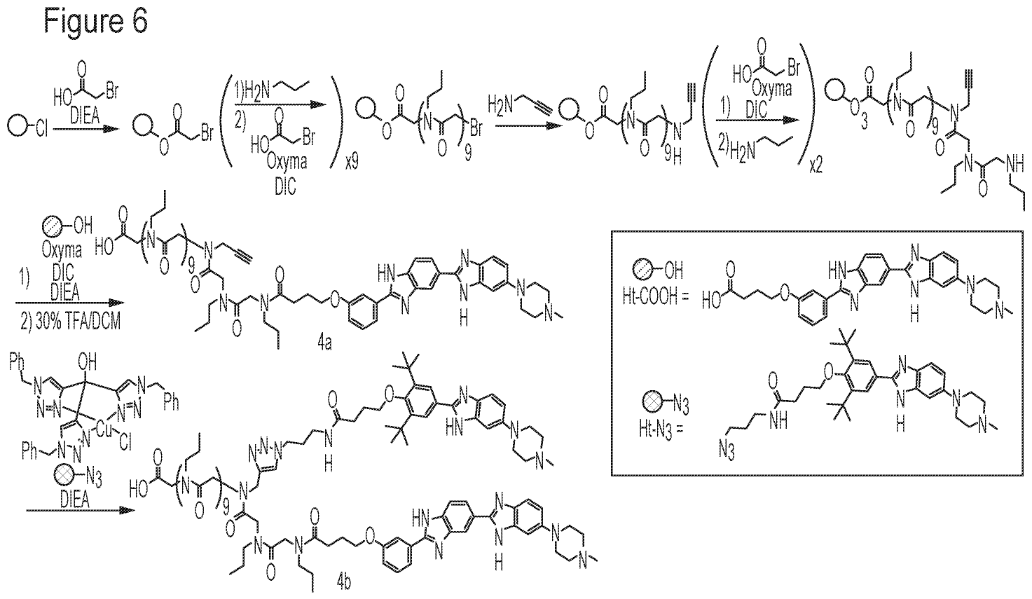

Synthesis of 4b. See FIG. 6. To a suspension of 2-Chlorotritylchloride resin (200 mg, 0.22 mmol) in DCM (0.9 mL) was added 4 N HCl in dioxane (0.3 mL) and the mixture was shaken at room temperature for 30 min. The resin was washed with DMF (3×3 mL) and DCM (3×3 mL). Then to the resin were added 1 M bromoacetic acid (1.1 mL) in DCM and DIEA (190 μL, 1.1 mmol). The mixture was shaken at room temperature for 2 h, and the resin was washed with DCM (3×3 mL) and DMF (3×3 mL). After addition of 1 mL of DMF and propylamine (180 μL, 2.2 mmol), the reaction was shaken at room temperature for 1 h. The resin was washed with DCM (3×3 mL) and DMF (3x×3 mL), and then 1 M bromoacetic acid (1.1 mL) in DMF, Oxyma (156 mg, 1.1 mmol) and DIC (169 μL, 1.1 mmol) were added. After shaking at room temperature for 2 h, the resin was washed with DCM (3×3 mL) and DMF (3×3 mL). This cycle was repeated eight additional times. Then to the resin were added 1.1 mL of DMF and propargylamine (141 μL, 2.2 mmol). The mixture was shaken at room temperature for 60 min, and the resin was washed with DCM (3×3 mL) and DMF (3×3 mL) followed by addition of 1 M bromoacetic acid (1.1 mL in DMF), Oxyma (156 mg, 1.1 mmol) and DIC (169 μL, 1.1 mmol). The mixture was shaken at room temperature for 2 h and the resin was washed with DCM (3×3 mL) and DMF (3×3 mL). Then to the resin were added 1.1 mL of DMF and propylamine (180 μL, 2.2 mmol). The mixture was shaken at room temperature for 60 min, and the resin was washed with DCM (3×3 mL) and DMF (3×3 mL). This cycle was repeated an additional time. Next, a mixture of Oxyma (85 mg, 0.6 mmol), DIC (93 μL, 0.6 mmol), DIEA (156 μL, 0.9 mmol) and Ht-COOH (225 mg, 0.45 mmol) in DMF (1.1 mL). The mixture was shaken at room temperature for 2 h and the resin was washed with DCM (3×3 mL) and DMF (3×3 mL). Then to the resin was added 30% TFA in DCM (2 mL) and the mixture was shaken at room temperature for 30 min. The mixture was filtered, and the filtrate was evaporated. To the residue was added Et2O and the precipitate was collected. The collected powder was purified using reverse phase HPLC as described in the General Synthetic Methods section to afford 4a (160 mg, 43% yield; C₈₉H₁₃₅N₁₈O₁₅ calculated mass: 1696.0354 (M+H)+; found: 1695.8215).

To 4a (100 mg, 0.059 mmol) were added stable copper (I) catalyst (8 mg, 0.013 mmol), Ht-N₃ (42 mg, 0.071 mmol), DIEA (200 μL, 1.2 mmol) and DMSO (1 mL). The reaction mixture was stirred at 60° C. for 6 h. Then to the reaction mixture was added acetonitrile (10 mL) and the precipitate was filtered. MeOH was added to the collected powder, and the mixture was filtered with filter paper. The filtrate was evaporated, and the residual powder was washed with acetonitrile to afford 4b as a yellow powder (22 mg, 0.0096 mmol, 16% yield). A portion of 4b was purified using reverse phase HPLC as described in the General Synthetic Methods section. C₁₂₂H₁₈₃N₂₆O₁₇ calculated mass: 2284.4255 (M+H)+; found: 2284.2666.

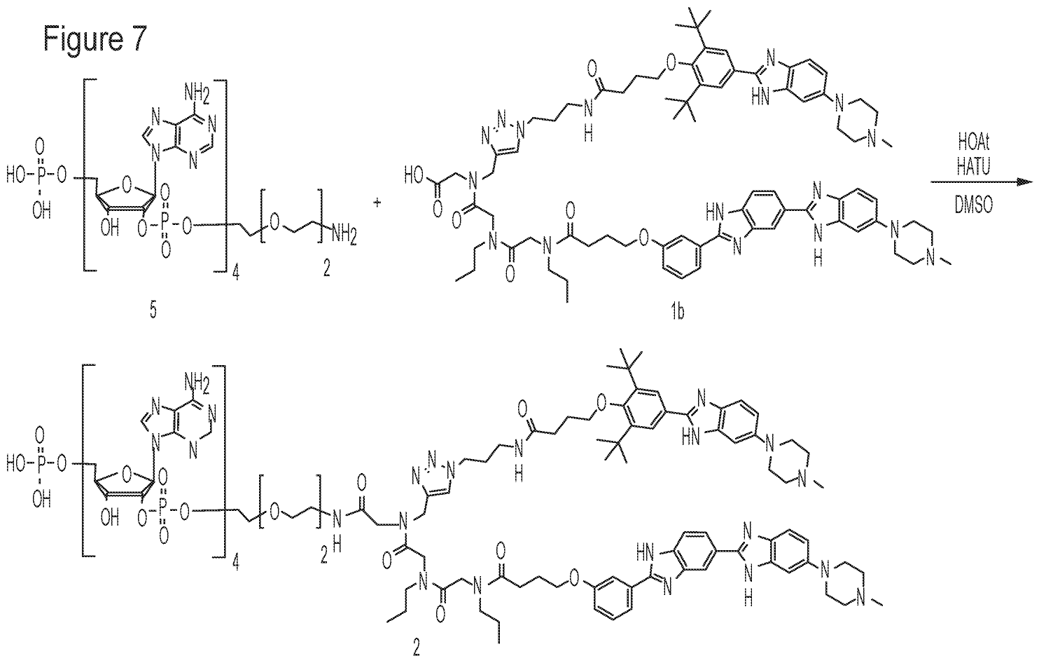

Synthesis of 2. See FIG. 7. To a 50 μL solution of 10 mM HOAt and 10 mM HATU in DMSO was added 50 μL of 10 mM 1b in DMSO (500 nmol). The mixture was shaken at room temperature for 2 h. Then to the mixture was added 106 μL of 0.94 mM 5 suspension in DMSO (100 nmol). After shaking for 1 h, 0.5 μL of a solution containing 400 mM HOAt and 400 mM HATU solution in DMSO was added to the mixture. After shaking for 15 h, an additional 0.5 μL of 400 mM HOAt and 400 mM HATU in DMSO was added to the mixture. After shaking for 24 h, 1.0 μL of 400 mM HOAt and 400 mM HATU solution in DMSO was added to the mixture. After shaking for 24 h, the reaction mixture was purified by reverse phase HPLC directly as described in the General Synthetic Methods section to afford 18 nmol of 2 (18% yield; C₁₂₃H₁₆₂N₃₈O₃₇P₅ calculated mass: 2918.0651 (M−H)−; found: 2917.8376).

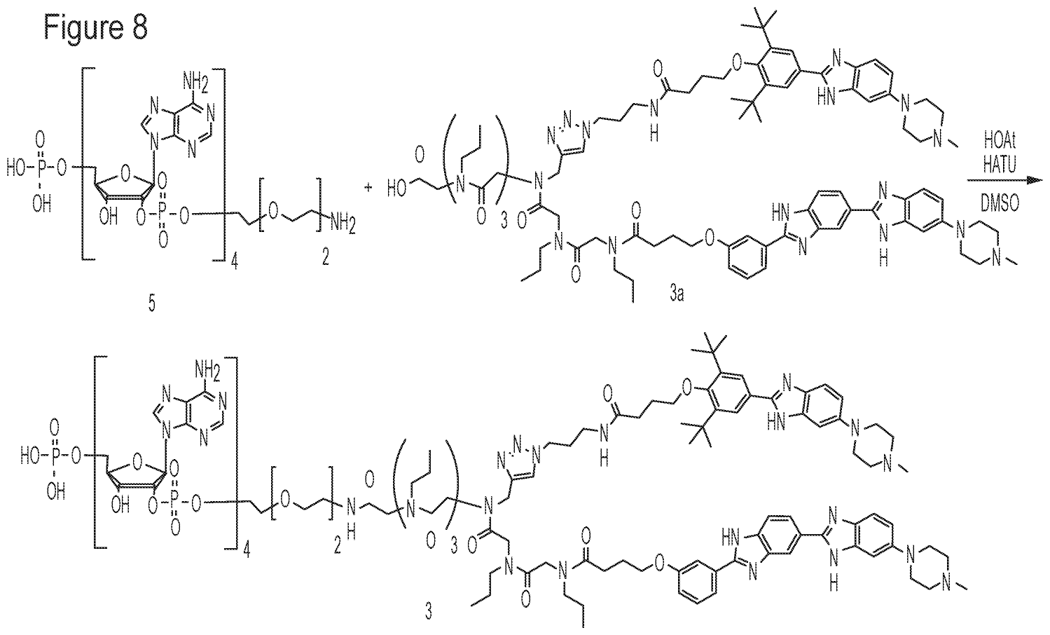

Synthesis of 3. See FIGS. 8. To 19 μL of 3a in DMSO (21 mM; 400 nmol) was added 1.0 μL of 400 mM HOAt and 400 mM HATU in DMSO. The mixture was shaken at room temperature for 30 min. Then 10 μL of 10 mM 5 in DMSO (100 nmol) was added. After shaking for 15 h, 1.0 μL of 400 mM HOAt and 400 mM HATU in DMSO was added to the mixture. After shaking for 7 h, 19 μL of 21 mM 3a solution in DMSO (400 nmol) and 1.0 μL of 400 mM HOAt and 400 mM HATU solution in DMSO were added to the mixture. After shaking for 16 h, the reaction mixture was purified by reverse phase HPLC directly as described in the General Synthetic Methods section to afford 2.4 nmol of 3 (2.4% yield; C₁₃₆H₁₈₉N₄₁O₄₀P₅ calculated mass: 3215.2704 (M−H)−; found: 3214.9058).

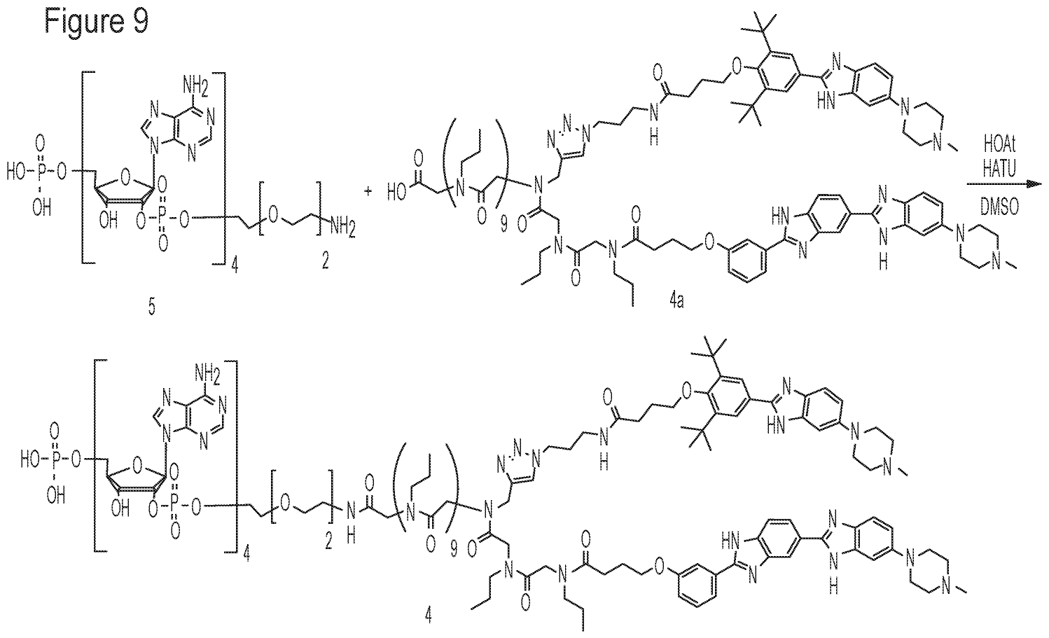

Synthesis of 4. See FIG. 9. To a 10 μL aliquot of 4b in DMSO (44 mM, 440 nmol) was added 1.0 μL of 400 mM HOAt and 400 mM HATU solution in DMSO. The mixture was shaken at room temperature for 10 min. Then 14 μL of 5 in DMSO (7.1 mM, 99 nmol) was added to the mixture. After shaking for 14 h, 10 μL of 4b solution in DMSO (3.3 mM, 33 nmol) and 1.0 μL of 400 mM HOAt and 400 mM HATU solution in DMSO were added to the mixture. After shaking for 24 h, the reaction mixture was purified by reverse phase HPLC directly as described in the General Synthetic Methods section to afford 2.6 nmol of 4 (2.6% yield; C₁₆₈H₂₄₃N₄₇O₄₆P₅ calculated mass: 3809.6808 (M−H)−; found: 3810.0334).

### Experimental Methods

Preparation of RNase L-GST protein: The pGEX-4T-RNaseL-GST plasmid was prepared as previously described 5 and kept in Storage Buffer (20 mM HEPES, pH 7.4, 70 mM NaCl, 2 mM MgCl₂).

In vitro RNase L oligomerization: An aliquot of 12 μM RNase L in RNase L Buffer (25 mM Tris-HCl (pH 7.4), 100 mM KCl, and 10 mM MgCl₂) was supplemented with fresh 7 mM β-mercaptoethanol and 50 μM of ATP. Dilutions of 2′-5′A₄, 1b, or 2 were prepared in RNase L Buffer supplemented with 7 mM β-mercaptoethanol and 50 μM of ATP and added to the solution of RNase L in a total volume of 17.4 μL. Solutions were incubated at room temperature for 5 min and then 1 μL of 44 mM dimethyl suberimidate (Thomas Scientific) in 0.4 M triethanolamine hydrochloride, pH 8.5, was added. After incubation at room temperature for 2 h, 3.6 μL of 6×Laemmli buffer (375 mM Tris-HCl, pH 6.8, 0.03% bromophenol blue, 0.6% β-mercaptoethanol, 12% SDS, 60% glycerol) was added.

After heating at 95° C. for 5 min, the samples were diluted 1:90 in 1×Laemmli buffer and a portion was resolved by SDS-PAGE. After transferring to a PVDF membrane, the membrane was blocked in 1×TBST [1×TBS with 0.1% Tween-20 (v/v)] containing 5% nonfat milk for 1 h. The membrane was incubated with RNase L antibody (1:5000 dilution; Cell Signaling Technology: D4B4J) overnight at 4° C. in 1×TBST containing 5% nonfat dry milk. The membrane was washed three times for 5 min each with 1×TBST and then incubated with 1:7000 anti-rabbit IgG horseradish peroxidase secondary antibody conjugate (Cell Signaling Technology: 7074S) in 1×TBST containing 5% nonfat dry milk for 1 h at room temperature. After washing five times for 5 min each with 1×TBST, protein levels were quantified by chemiluminescence with SuperSignal West Pico Chemiluminescent Substrate (Pierce Biotechnology) per the manufacturer's protocol. Protein band signals were quantified using ImageJ software (National Institutes of Health).

Determination of RNA cleavage by fluorescence: A model RNase L substrate RNA1 labeled with a 5′ 6-Fluorescein and 3′ Iowa Black® FQ (5′-6FAM-UUAUCAAAU UCUUAUUUGCCCCAUUUUUUUGGUUUA 3′ (SEQ ID NO: 1)—Iowa Black® FQ: RNA 1) was purchased from Integrated DNA Technologies, Inc. (IDT), which also HPLC purified the oligonucleotide. A model of pri-miR-96 RNA labeled with 5′ 6-Fluorescein and 3′ Quencher (IQ4, structure undisclosed) (5′-6FAM UGGCCGAUUUUGGCACUAGCAC AUUUUUGCUUGUGUCUCUCCGCUCUGAGCAAUCAUGUGCAGUGCCAAUAUGGGAAA 3′ (SEQ ID NO: 2)—IQ4: RNA 2) was purchased from and HPLC purified by Chemgenes. Solutions of RNA 1 or RNA 2 were folded at 70° C. for 5 min and cooled to room temperature in RNase L Buffer without MgCl2, β-mercaptoethanol or ATP. After cooling, the RNA was supplemented with 10 mM MgCl2, fresh 7 mM β-mercaptoethanol, and 50 μM of ATP. Samples of 2′-5′A+RNase L (10 nM) or 2+RNase L (100 nM) were prepared in 1×RNase L Buffer and incubated at 4° C. for 30 min. To these samples were added 100 nM of folded RNA 1 or RNA 2. The samples were transferred to Corning non-binding surface half area 96-well black plates, and incubated at room temperature for 60 min. Fluorescence intensity (Ex: 485 nm, Em: 528 nm) was measured on a BioTek FLx800 plate reader. Relative Fluorescence Enhancement was calculated by normalizing the fluorescent signal of treated RNA samples to the fluorescent signal of untreated RNA samples. Percentage RNA Cleavage was calculated by normalizing sample fluorescent signals relative to the maximum fluorescent signal, set as 100%. For experiments with tRNA competition, tRNA from brewer's yeast (Roche) was phenol-chloroform extracted. Dilutions of folded tRNA were prepared in RNase L Buffer. Folded RNA 2 was then added to the solution and the experiment was completed as described above. Normalized RNA Cleavage was calculated by normalizing sample fluorescent signals relative to the maximum fluorescent signal (compound with no tRNA), set as 1.

PCR amplification & in vitro transcription: The DNA template for miR-96 primary transcript RNA (pri-miR-96) (5′—GGGTGGCCGATTTTGGCACTAGCACATTTTT GCTTGTGTCTCTCCGCTCTGAGCAATCATGTGCAGTGCCAATATGGGAAA) (SEQ ID NO: 3) was purchased from IDT with standard desalting and used without further purification. This template was PCR amplified in 1×PCR Buffer (10 mM Tris, pH 9.0, 50 mM KCl, and 0.1% (v/v) Triton X-100), 2 μM forward primer (5′—GGCCGGATCCTAATACGACTCACTA TAGGGTGGCCGATTTTGGC) (SEQ ID NO: 4), 2 μM reverse primer (5′—TTTCCCATATTGGCA) (SEQ ID NO: 5), 4.25 mM MgCl2, 330 μM dNTPs, and 1 μL of Taq DNA polymerase in a 50 μL reaction. PCR cycling conditions were initial denaturing at 95° C. for 90 s, followed by 25 cycles of 95° C. for 30 s, 55° C. for 30 s, and 72° C. for 60 s.

The DNA templates for the RNA cassette displaying the pri-miR-96 Drosha site motifs (RNA 3) (5′—GGGAGAGGGTTTAATCCGATTTTGGTACGAAAGTACCAATAT GGGATTGGATCCGCAAGG) (SEQ ID NO: 6) and the RNA cassette where the pri-miR-96 Drosha site motifs are base paired (RNA 4) (5′—GGGAGAGGGTTTAATCCGATTT TGGTACGAAAGTACCAAAATCGGATTGGATCCGCAAGG) (SEQ ID NO: 7) were purchased from IDT with standard desalting and used without further purification. These templates were PCR amplified in 1×PCR Buffer (10 mM Tris, pH 9.0, 50 mM KCl, and 0.1% (v/v) Triton X-100), 2 μM cassette forward primer (5′—GGCCGAATTCTAATACGACTCACTATAGG GAGAGGGTTTAAT) (SEQ ID NO: 8), 2 μM cassette reverse primer (5′—CCTTGCGGATCCAAT) (SEQ ID NO: 9), 4.25 mM MgCl2, 330 μM dNTPs, and 1 μL of Taq DNA polymerase in a 50 μL reaction. PCR cycling conditions were initial denaturing at 95° C. for 90 s, followed by 25 cycles of 95° C. for 30 s, 50° C. for 30 s, and 72° C. for 60 s.

RNA was in vitro transcribed, using 300 μL of the PCR product, by T7 RNA polymerase in 1×Transcription Buffer (40 mM Tris-HCl, pH 8.1, 1 mM spermidine, 0.001% (v/v) Triton X-100 and 10 mM DTT) with 2.25 mM of each rNTP and 5 mM MgCl2 at 37° C. for 18 h. The RNA was then purified on a denaturing 15% polyacrylamide gel and isolated as previously described. RNA concentration was determined by UV absorbance at 260 nm at 90° C. using a Beckman Coulter DU800 UV-Vis spectrophotometer with a Peltier temperature controlling unit. Extinction coefficients were calculated using the Oligo Extinction Coefficient Calculator.

RNA mapping experiments: RNA mapping was performed on in vitro transcribed pri-miR-96. RNA that was 5′ end labeled with ³²P as previously described. RNA was folded at 95° C. for 30 s and cooled to room temperature. Samples were prepared in RNase L buffer without MgCl2, β-mercaptoethanol or ATP, and then supplemented with 10 mM MgCl₂, fresh 7 mM β-mercaptoethanol and 50 μM of ATP after folding. Aliquots of 2′-5′A₄ or 2 were diluted in RNase L Buffer, and then ˜4000 counts of folded radioactively labeled pri-miR-96 RNA was added. Samples were incubated for 30 min at room temperature followed by addition of RNase L at an equimolar concentration of 2′-5′Ae4 or 2. The samples incubated for 60 min at room temperature and then quenched by addition of an equal volume of 2×Loading Buffer (8 M urea, 20 mM EDTA, 2 mM Tris-base, 0.01% bromophenol blue and 0.01% xylene cyanol). For competition with 1a, samples were prepared as described above, except serial dilutions of 1a were added in addition to constant concentrations of 2′-5′A₄ or 2 and incubated with pri-miR-96 RNA for 15 min at room temperature before adding RNase L at the appropriate concentration. Samples were incubated for 60 min at room temperature. A base hydrolysis ladder was generated by incubating RNA at 95° C. in 1×Hydrolysis Buffer (50 mM NaHCO₃, 1 mM EDTA, pH 9.4) for 1.5 min. To identify all G residues, pri-miR-96 was incubated with 20 U of T1 ribonuclease (ThermoFisher Scientific) in 1×Denaturing T1 buffer (25 mM sodium citrate, pH 5, 7 M urea, 1 mM EDTA) for 20 min. RNA fragments were resolved on a denaturing 12.5% polyacrylamide gel and quantified by phosphorimaging and QuantityOne (BioRad). Percentage Counts Relative to Full Length was calculated by dividing the counts quantifying the appropriate band (Full length band, U12 band, or U35 band) by the counts upon quantifying the full lane and multiplying by 100.

Measurement of binding affinities: Dissociation constants for the binding of nucleic acids to compounds were determined using an in-solution, fluorescence-based binding assay. Similar binding assays were used to assess the binding affinity of the parent compound, 1a, to the Drosha site of pri-miR-96. Fluorescence from this assay is derived from the intrinsic fluorescence of the compound (Ex: 345 nm, Em: 460 nm). Upon binding to RNA, the fluorescence of the compound increases, allowing the generation of binding dissociation curves with the appropriate RNA. Nucleic acids were folded in 1×Crowded Binding Buffer (8 mM Na2HPO4, 190 mM NaCl, 1 mM EDTA, and 40 μg/mL BSA in 20% (w/v) PEG8000) by heating at 60° C. for 5 min and then cooled to room temperature. Compounds were added to a final concentration of 500 nM. Next, 1:2 serial dilutions of RNA were performed in 1×Crowded Binding Buffer supplemented with 500 nM of compound. Solutions were incubated for 30 min and then transferred to Corning non-binding surface half area 96-well black plates. Fluorescence intensity (Ex: 345 nm, Em: 460 nm) was then measured on a Molecular Devices SpectraMax M5 plate reader. Change in fluorescence intensity was fit as a function of RNA concentration with equation 1 (Eq 1):

I=I₀+0.5Δε(([FL]₀+[RNA]₀+Kₜ)−([FL]₀+[RNA]₀+Kₜ)²−4[FL]₀[RNA]₀)^{0.5}) (Eq 1)

where I and I₀ are the observed fluorescence intensity in the presence and absence of nucleic acid, respectively, Δε is the difference between the fluorescence intensity in the absence and in the presence of infinite nucleic acid concentration, [FL]₀ and [RNA]₀ are the concentrations of compound and nucleic acid, respectively, and Kₜ is the dissociation constant.

Cell culture: All cells were maintained at 37° C. with 5% CO2. MDA-MB-231 (HTB-26, ATCC) cells were cultured in RPMI 1640 medium with L-glutamine & 25 mM HEPES (Corning) supplemented with 10% FBS (Sigma) and 1×Antibiotic-Antimycotic (Corning). A549 (CCL-185, ATCC), HeLa (CCL-2, ATCC), and MCF7 (HTB-22, ATCC) cells were cultured in DMEM medium with 4.5 g/L glucose (Corning), supplemented with 10% FBS (Sigma), 1×Glutagro (Corning), and 1×Antibiotic-Antimycotic (Corning). MCF10a (CRL-10317, ATCC) cells were cultured in DMEM/F12 50/50 with L-glutamine & 15 mM HEPES (Corning), supplemented with 10% FBS (Sigma), 20 ng/mL human epidermal growth factor (Pepro Tech Inc.), 0.5 mg/mL hydrocortisone (Pfaltz & Bauer), 100 ng/mL cholera toxin (Sigma-Alrich), 10 μg/mL insulin (Sigma-Aldrich), and 1×Antibiotic-Antimycotic (Corning). For treatment of compounds, stocks were diluted in growth media and added to cells for 24-72 h. For transfection of the 2′-5′ A₄ oligonucleotide and plasmid DNA to overexpress either miR-96 hairpin precursor (GeneCopoeia: HmiR0116-MR04) or RNase L (pcDNA3-RNaseL; R. H. Silverman, Cleveland Clinic) 7 in 24- or 96-well plates, Lipofectamine 2000 was used according to the manufacturer's protocol. After transfection, the medium was removed and replaced with growth medium containing compound as described above. For transfection of a control (Santa Cruz Biotechnology: sc-37007) or RNase L targeting siRNA (Santa Cruz Biotechnology: sc-45965), Lipofectamine RNAiMAX Reagent was used according to the manufacturer's protocol.

RNA isolation and RT-qPCR: Total RNA was extracted from untreated and treated cells by using a Quick-RNA MiniPrep (Zymo Research) per the manufacturer's protocol. Approximately 200-600 ng of total RNA was used in subsequent reverse transcription reactions using a miScript II RT Kit (Qiagen) per the manufacturer's recommended protocols. RT-qPCR primers were purchased from Eurofins or IDT and used without further purification. The RT-qPCR samples were prepared using Power SYBR Green PCR Master Mix (Applied Biosystems) and completed on a 7900HT Fast Real Time PCR System (Applied Biosystems). RNA expression levels were determined using the ΔΔCt method and normalized with U6 small nuclear RNA as a housekeeping gene. Upon treatment with chimeric compound 2, relative cleavage levels were calculated according to equation 2 (Eq. 2) in order to control for the effect of parent compound 1a, on pri-miR-96 expression:

Relative RNA Cleavage=(Relative RNA Expression with 1a treatment)/(Relative RNA Expression 2 treatment) (Eq. 2)

RNA Immunoprecipitation: MDA-MB-231 cells were grown in 6-well plates to ˜70% confluency and treated with either 200 nM of 2′-5′A₄ or 200 nM of 2 for 48 h. Cells were washed with 1×DPBS, removed from the plate with Accutase (Innovative Cell Technologies, Inc.), and washed with ice-cold 1×DPBS. Cells were then lysed in 100 μL of M-PER buffer supplemented with 80 U RNaseOUT Recombinant Ribonuclease Inhibitor (Invitrogen) and 1×Protease Inhibitor Cocktail III for Mammalian Cells (Research Products International Corp.) according to the manufacturer's instructions. The samples were centrifuged at 13000×g, and supernatants were incubated overnight at 4° C. with Dynabeads Protein A (Life Technologies) that were bound to either β-actin mouse primary antibody (Cell Signaling: 8H10D10) or RNase L mouse primary antibody (Santa Cruz Biotechnology: sc-74405). After incubation, beads were washed three times with 1×DPBS with 0.02% Tween-20 after which RNA was extracted using a miRNeasy Mini Kit (Qiagen) according to manufacturer's instructions. Glycogen (20 μg) was added before addition of ethanol to aid in RNA precipitation. RT-qPCR was completed as described above. Relative RNA expression levels were determined by the ΔΔCt method and normalized to 18S rRNA as a housekeeping gene. Normalized fold change was calculated by dividing relative expression levels of the gene of interest in the cDNA library prepared from RNA extracted from the RNase L immunoprecipitated fraction by the relative expression levels of the gene of interest in the cDNA library prepared from RNA extracted from the β-actin immunoprecipitated fraction, or equation 3 (Eq. 3):

Normalized Fold Change=(Relative RNA Expression in RNase L fraction)/(Relative RNA Expression in β-actin fraction) (Eq. 3)

FOXO1 Western blot: MDA-MB-231 cells were grown to ˜60% confluency in 6-well plates. Cells were incubated with 20 or 200 nM of 2 for 48 h. Total protein was extracted using M-PER Mammalian Protein Extraction Reagent (Pierce Biotechnology) supplemented with 1×Protease Inhibitor Cocktail III for Mammalian Cells (Research Products International Corp.) per the manufacturer's protocol and quantified using a Micro BCA Protein Assay Kit (Pierce Biotechnology). A 30 μg aliquot of total protein was resolved on an 8% Bis-Tris SDS-polyacrylamide gel and then transferred to a PVDF membrane. The membrane was then blocked in 5% (w/v) nonfat dry milk dissolved in 1×TBST for 1 h at room temperature. The membrane was then incubated with 1:2000 rabbit mAb FOXO1 primary antibody (Cell Signaling Technology: C29H4) in 1×TBST containing 5% nonfat dry milk overnight at 4° C. The membrane was washed five times for 5 min each with 1×TBST and then incubated with 1:2000 anti-rabbit IgG horseradish peroxidase secondary antibody conjugate (Cell Signaling Technology: 7074S) in 1×TBST containing 5% nonfat dry milk for 1 h at room temperature. After washing seven times for 5 min each with 1×TBST, protein levels were quantified by chemiluminscence with SuperSignal West Pico Chemiluminescent Substrate (Pierce Biotechnology) per the manufacturer's protocol. The membrane was stripped with 1×Stripping Buffer (200 mM glycine, pH 2.2, 1% Tween-20 and 0.1% SDS) two times for 5 min each, followed by washing 3 times in 1×TBST. Then, the membrane was blocked and probed for β-actin following the same procedure described above using 1:5000 mouse β-actin primary antibody (Cell Signaling Technology: 8H10D10). The membrane was washed five times with 1×TBST and incubated with 1:10000 anti-mouse IgG horseradish-peroxidase secondary antibody conjugate (Cell Signaling Technology: 7076S). After washing seven times with 1×TBST for 5 min each, protein levels were quantified as described above. ImageJ software was used to quantify band intensities.

Flow cytometry: Cells were grown in 6-well plates to ˜60% confluency and then incubated with dilutions of compounds (1a or 2) for 72 h. Alternatively, cells were transfected with a plasmid overexpressing a hairpin precursor of miR-96 (GeneCopoeia: HmiR0116-MR04) at 60% confluency using JetPRIME transfection reagent (Polyplus transfection) according to the manufacturer's protocol for 5 h and then the medium was changed and treated with compound as described above. Cells were removed from the plate using Accutase (Innovative Cell Technologies, Inc.) and washed twice with ice-cold 1×DPBS and 1×Annexin Binding Buffer (50 mM HEPES, pH 7.4, 700 mM NaCl and 12.5 mM CaCl2). Cells were re-suspended in 100 μL 1×Annexin Binding Buffer containing 5 μL Annexin V-APC (BD Pharmigen). The solution was incubated for 10 min at room temperature followed by washing twice with 1×Annexin Binding Buffer. Cells were then stained with 1 μg/mL propidium iodide (Sigma Aldrich) in 300 μL of 1×Annexin Binding Buffer for 10 min at room temperature. Flow cytometry was performed using a BD LSRII instrument (BD Biosciences). Compound uptake was measured by reading compound fluorescence upon excitation with a DAPI-UV laser. Gated viable cells were then analyzed for the compound uptake by taking the mean values in a DAPI-UV histogram and normalizing untreated and 1a as 0% and 100%, respectively. At least 10,000 events were used for analysis.

Analysis of compound stability: MDA-MB-231 cells were grown to 80% confluency in a 24-well plate and treated with 5 μM of 2. After 24 h of incubation, the medium was removed and the cells were washed with 1×DPBS. Cells were lysed by adding 400 μL of Nanopure water and freezing at −80° C. overnight. After thawing, samples were centrifuged at 13000×g. The supernatant was removed and dried down completely in a Labconco SpeedVac Concentrator. Acetonitrile (200 μL) was added and samples were centrifuged at 13000×g, after which the supernatant was removed and dried down. Samples were dissolved in 20 μL of water, and 10 μL was purified using a ZipTip with 0.6 μL of C18 resin (EMD Millipore). Compound was eluted in 50% acetonitrile/50% Nanopure water. Compound detection by MALDI-TOF mass spectral analysis was performed as described in the General Synthetic Methods section. The total ion counts of the intact compound (m/z=2919) and of the fragment 2′-5′ A₄ (m/z=1544) observed upon MALDI mass spectrometry were collected; the major mode of putative metabolism is the amide bond between 2 and 2′-5′ A₄ thus a change in this ratio could indicate metabolism. Percent compound intact was calculated as the ratio of the total ion counts of the intact compound to the total ion counts of the 2′-5′ A₄ fragment, where the ratio from the stock of compound 2 was normalized to 100%.

Caspase 3/7 activity measurements: Approximately 5,000 MDA-MB-231 or MCF10a cells were plated into black, cell-culture treated, 96-well plates (Corning). At ˜60% confluency, cells were treated for 48 h with dilutions of 2 in appropriate growth medium. Caspase 3/7 activity was measured using a Caspase-Glo 3/7 kit (Promega) according to the manufacturer's protocol. Fold change in Caspase activity was calculated by normalizing treated samples to the untreated samples after subtracting background sample values.

Measurement of Catalytic Activity: MDA-MB-231 cells were plated into a 6-well plate (Corning). At 80% confluency, the medium was aspirated and the monolayer was washed with 1×DPBS. Dilutions of 2 in cell culture media were added to the cells and incubated for 24 h. Cells were removed from the plate using Accutase (Innovative Cell Technologies, Inc.) and washed with 1×DPBS. Cells were lysed using 200 μL of RNA lysis buffer from a Quick-RNA MiniPrep (Zymo Research). An aliquot of 50 μL was transferred to Corning non-binding surface half area 96-well black plates. Fractions of untreated cell lysate were combined and used to generate a standard curve of 2, by spiking in known concentrations of 2 (50 nM, 100 nM, 250 nM, 500 nM, 1000 nM). Fluorescence intensity (Ex: 345 nm, Em: 460 nm) was then measured on a Molecular Devices SpectraMax M5 plate reader. The concentration of 2 in the 50 μL aliquot was extrapolated using the generated standard curve, and the amount of 2 (pmol) in the full 200 μL volume was then calculated.

RNA from the samples was then isolated and RT-qPCR proceeded as described above, with standard curves using in vitro transcribed pri-miR-96 (10 ng, 1 ng, 0.1 ng, 0.01 ng, 0.001 ng, 0.0001 ng, 0 ng) being included with each run in order to accurately calibrate the Ct values. The amount of cleaved pri-miR-96 was then calculated by taking the difference between the pmol of pri-miR-96 in untreated samples and the pmol of pri-miR-96 in 2 treated samples. Catalytic activity, or turnover, was then calculated by taking the ratio of the pmol of cleaved pri-miR-96 and the pmol of 2 in the sample.

### TGP-210-RL Studies

### Experimental Model and Subject Details

MDA-MB-231 (HTB-26; ATCC) cells were cultured in Roswell Park Memorial Institute (RPM I) 1640 media with L-glutamine & 25 mM HEPES (Corning) supplemented with 10% fetal bovine serum (FBS) (Sigma) and 1×Penicillin Streptomycin Solution (Corning). Cells cultured in normoxia were maintained at 37° C. in ambient atmosphere (˜21% O₂) with 5% CO₂. Cells cultured in hypoxia were maintained at 37° C., <1% O₂ in a nitrogen filled hypoxic chamber (Billups-Rothenberg, Inc.), and 5% CO₂. Cells were directly purchased from ATCC, but were not authenticated.

For compound treatment (TGP-210, TGP-210-RL, LNA-210, Scr-LNA), compound stocks in DMSO or water were diluted in growth medium and added to cells for 24-48 h. For transfection of cells with plasmids (miR-210 overexpression plasmid or RNase L overexpression plasmid) Lipofectamine 2000 (Invitrogen) was used according to the manufacturer's protocol. For transfection of cells with siRNAs (control siRNA-A or RNase L siRNA) or 2′-5′ A₄, Lipofectamine RNAiMAX Reagent (Invitrogen) was used according to the manufacturer's protocol.

### Method Details

### In Vitro Fluorescent RNA Cleavage

A miR-210 Hairpin Precursor RNA labeled with a 5′ 6-Fluorescein (6FAM) and 3′ Black Hole Quencher (IQ4) (5′—6FAM AGCCCCUGCCCACCGCACACUGCGCUGCCCCAGACCCACUGU—GCGUGUGACAGCGGCU—3′ (SEQ ID NO: 10) IQ4; 5′ FAM/3′ BHQ miR-210 Hairpin Precursor RNA) was purchased from Chemgenes with HPLC purification. Solutions of 5′ FAM/3′ BHQ miR-210 Hairpin Precursor RNA (100 nM) were folded at 60° C. for 5 min and slowly cooled to room temperature in 1×RNase L Buffer (25 mM Tris-HCl, pH 7.4, 100 mM KCl) without MgCl₂, β-mercaptoethanol or ATP. After folding, the RNA was supplemented with 10 mM MgCl₂, fresh 7 mM β-mercaptoethanol, and 50 μM of ATP. Next, 50 nM of RNase L, prepared as described previously, and 100 nM of compounds (TGP-210-2′-5′ Aₙ derivatives, where n=2−4) were prepared in 1×RNase L Buffer and added to the RNA. The samples were then transferred to Corning non-binding surface half area 96-well black plates. The samples were incubated at room temperature for the defined time points (15, 30, 60, 120, and 720 min) after which the fluorescence intensity (Ex: 485 nm, Em: 525 nm) was measured using a SpectraMax M5 plate reader. The percentage change in fluorescence intensity, where enhancement of fluorescence intensity was indicative of RNA cleavage, was determined by calculating the percentage change in sample fluorescent signals relative to the untreated fluorescent signal.

### In Vitro RNA Cleavage Mapping

Alternatively, a 5′-³²P end labeled miR-210 hairpin precursor was in vitro transcribed as described previously. Aliquots of TGP-210-RL were diluted in RNase L Buffer, and then ˜5000 counts of folded 5′-³²P end labeled pre-miR-210 RNA were added. Samples were incubated for 30 min at room temperature followed by addition of RNase L at an equimolar concentration of TGP-210-RL. The samples were incubated for 60 min at room temperature and then quenched by addition of an equal volume of 2×Loading Buffer (8M urea, 20 mM EDTA, 2 mM Tris-base, 0.01% bromophenol blue and 0.01% xylene cyanol). A base hydrolysis ladder was generated by incubating RNA at 95° C. in 1×Hydrolysis Buffer (50 mM NaHCO₃, 1 mM EDTA, pH 9.4) for 5 or 10 min. To identify all G residues, pre-miR-210 was incubated with dilutions of T1 ribonuclease (ThermoFisher Scientific) in 1×Denaturing T1 Buffer (25 mM sodium citrate, pH 5, 7 M urea, 1 mM EDTA) for 20 min at room temperature. RNA fragments were resolved on a denaturing 15% polyacrylamide gel and imaged by phosphorimaging and QuantityOne software (BioRad).

### In Vitro RNase L Oligomerization

An aliquot of RNase L (3 μM) in 1×RNase L Buffer was supplemented with 10 mM MgCl₂, fresh 7 mM β-mercaptoethanol, and 50 μM of ATP. Dilutions of 2′-5′ A₄, TGP-210, or TGP-210-RL were prepared in 1×RNase L Buffer supplemented with 10 mM MgCl₂, fresh 7 mM β-mercaptoethanol, and 50 μM of ATP and added to the solution of RNase L in a total volume of 17.4 μL. The RNase L/compound solutions were incubated at room temperature for 5 min and then 1 μL of 44 mM dimethyl suberimidate (Thomas Scientific) in 0.4 M triethanolamine hydrochloride, pH 8.5, was added. After incubating at room temperature for 2 h, 3.6 μL of 6×Laemmli buffer (375 mM Tris-HCl, pH 6.8, 0.03% bromophenol blue, 0.6% β-mercaptoethanol, 12% SDS, 60% glycerol) was added. After heat denaturing the samples at 95° C. for 5 min, the samples were diluted 1:90 in 1×Laemmli buffer and then resolved by SDS-PAGE. After transferring to a PVDF membrane, the membrane was blocked in 1×TBST [1×TBS with 0.1% Tween-20 (v/v)] containing 5% nonfat milk for 1 h. The membrane was incubated with RNase L antibody (1:5000 dilution; Cell Signaling Technology: D4B4J) overnight at 4° C. in 1×TBST containing 5% nonfat dry milk. The membrane was washed three times for 5 min each with 1×TBST and then incubated with 1:10000 anti-rabbit IgG horseradish peroxidase secondary antibody conjugate (Cell Signaling Technology: 7074S) in 1×TBST containing 5% nonfat dry milk for 2 h at room temperature. After washing the membrane five times for 5 min each with 1×TBST, protein levels were quantified by chemiluminescence with SuperSignal West Pico Chemiluminescent Substrate (Pierce Biotechnology) per the manufacturer's protocol. RNase L bands associated with monomeric or oligomeric signals were quantified using ImageJ software (National Institutes of Health).

### Microscale Thermophoresis (MST) Binding Measurements

MST fluorescent measurements were performed on a Monolith NT.115 system (NanoTemper Technologies) using the fluorescence of a 5′-Cy5 labeled miR-210 Hairpin RNA (5′—Cy5 CGCACACUGCGCUGCCCCAGACCCACUGUGCG) (SEQ ID NO: 11), a 5′-Cy5 miR-210 Mutant RNA (5′—Cy5 CGCACAGUGCGCUGCCCCAGACCCACUGUGCG) (SEQ ID NO: 12), or a 5′ Cy5 DNA Hairpin (5′—Cy5 CGCGAATTCGCGTTTTCGCGAATTCGCG) (SEQ ID NO: 13) which were purchased from IDT with RNase-free HPLC purification and used without further purification. The RNA (5 nM) was prepared in 1×MST Buffer (8 mM Na₂HPO₄, 190 mM NaCl, 1 mM EDTA, and 0.05% (v/v) Tween-20) and folded by heating at 60° C. for 5 min, and slowly cooling to room temperature. Compounds (TGP-210 or TGP-210-RL) were diluted in 1×MST Buffer and then were added to a final concentration of 20 μM, followed by 1:2 dilutions in 1×MST Buffer containing 5 nM RNA. Alternatively, RNase L in 1×MST Buffer was added to a final concentration of 50 nM in addition to compound and RNA. Samples were incubated for 30 min at room temperature and then loaded into premium-coated capillaries (NanoTemper Technologies). Fluorescence measurements (Ex: 605-645 nm, Em: 680-685 nm) were performed at 20% LED and 80% MST power, with a Laser-On time of 30 s and Laser-Off time of 5 s. The data were analyzed by thermophoresis analysis and fitted by the quadratic binding equation in MST analysis software (NanoTemper Technologies). Dissociation constants were then determined by curve fitting using a single-site model.

### Cellular Uptake by Flow Cytometry

The MDA-MB-231 cells were grown in 6-well plates to ˜60% confluency and then incubated with dilutions of TGP-210 or TGP-210-RL for 48 h. Cells were removed from the plate using Accutase (Innovative Cell Technologies, Inc.) and washed twice with ice-cold 1×DPBS. Upon re-suspending ˜1×10⁶ cells in 1×DPBS, compound uptake was measured by reading compound fluorescence upon excitation with a DAPI-UV laser. Gated viable cells were then analyzed for the compound uptake by taking the mean count values of samples in a DAPI-UV histogram and normalizing untreated and TGP-210 samples as 0% and 100%, respectively. At least 10,000 events were used for analysis.

### Cellular Uptake by Confocal Microscopy

The MDA-MB-231 cells were grown to ˜80% confluence in a Mat-Tek 96-well glass bottom plates in growth medium. Cells were treated with 5000 nM of TGP-210 or TGP-210-RL in complete growth medium for 24 h under hypoxic conditions. The growth medium was removed and cells were then washed with 1×DPBS and fixed with 4% paraformaldehyde in 1×DPBS at 37° C. and 5% CO₂ for 10 minutes. The cells were then washed twice with 1×Hank's Balanced Salt Solution (HBSS) and a 1:10000 dilution of SYTO 82 nuclear stain in 1×HBSS was added. After 10 min of incubation at room temperature, cells were washed three times in 1×HBSS and resuspended in 100 μL of 1×HBSS and intrinsic TGP-210 or TGP-210-RL fluorescence (DAPI channel) or SYTO 82 fluorescence (TRITC channel) were imaged using an Olympus FluoView 1000 confocal microscope at 40× magnification.

### RNA Isolation and RT-qPCR

MDA-MB-231 cells were treated in normoxic or hypoxic conditions for 24-48 h, as described above in the Experimental Model and Subject Details section. Total RNA was extracted from cells by using a Quick-RNA MiniPrep (Zymo Research) according to the manufacturer's protocol. Subsequent reverse transcription reactions were completed on approximately 200-600 ng of total RNA using a miScript II RT Kit (Qiagen) according to the manufacturer's protocol. The RT-qPCR samples were prepared using Power SYBR Green PCR Master Mix (Applied Biosystems) and completed on a 7900HT Fast Real Time PCR System (Applied Biosystems) according to the manufacturer's protocol. RT-qPCR primers were purchased from Eurofins or IDT and used without further purification. RNA expression levels were determined using the ΔΔCₜ method and normalized using 18S ribosomal RNA or U6 small nuclear RNA as housekeeping genes. For qPCR miRNA profiling, a custom panel of miRNAs based on Qiagen's MHS-001Z Gene Table miRNA profiling plate was used. Downstream analysis was performed using the miScript miRNA PCR Array template Version 1.1 using an adjusted version of the MHS-001Z Gene Table. Data were normalized using SNORD44 and RNU6 as housekeeping genes. Further data analysis, processing, and statistics were performed in the GraphPad Prism software. Upon treatment with chimeric compound TGP-210-RL, relative cleavage levels were calculated according to equation 1A (Eq. 1A) in order to control for the effect of parent compound TGP-210, on pre-miR-210 expression:

$$Relative RNA Cleavage = \frac{\begin{matrix}Relative RNA Expression with TGP - \\ 210 - RL treatment\end{matrix}}{Relative RNA Expression TGP - 210 treatment} \qquad ( Eq . 1 A )$$

### RNA Immunoprecipitation

MDA-MB-231 cells were grown in 6-well plates to ˜70% confluency and treated with 200 nM of 2′-5′ A₄ or 200 nM TGP-210-RL diluted in cell media for 48 h under hypoxic conditions. The cell monolayer was washed with 1×DPBS, removed from the plate with Accutase (Innovative Cell Technologies, Inc.), and washed with ice-cold 1×DPBS. Cells were lysed in 100 μL of M-PER buffer supplemented with 80 U RNaseOUT Recombinant Ribonuclease Inhibitor (Invitrogen) and 1×Protease Inhibitor Cocktail III for Mammalian Cells (Research Products International Corp.) according to the manufacturer's instructions. The samples were centrifuged at 13000×g for 15 min and supernatant was removed. Supernatants were incubated overnight at 4° C. with Dynabeads Protein A (Life Technologies) that were bound to either β-actin mouse primary antibody (Cell Signaling Technologies; 3700S) or RNase L mouse primary antibody (Santa Cruz Biotechnology; sc-74405). After antibody incubation, the beads were washed three times with 1×DPBS supplemented with 0.02% Tween-20 and then total RNA was extracted from beads using a miRNeasy Mini Kit (Qiagen) according to manufacturer's instructions. Glycogen (20 μg) was added before addition of ethanol to aid in RNA precipitation. RT-qPCR was completed as described above. Relative RNA expression levels were determined by the ΔΔCₜ method and normalized to 18S rRNA as a housekeeping gene. Normalized fold change was calculated by dividing relative expression levels of the gene of interest in the cDNA library prepared from RNA extracted from the RNase L immunoprecipitated fraction by the relative expression levels of the gene of interest in the cDNA library prepared from RNA extracted from the β-actin immunoprecipitated fraction, or equation 2A (Eq. 2A):

$$Normalized Fold Change = \frac{Relative RNA Expression in RNase L fraction}{Relative RNA Expression in β \text{-} actin fraction} \qquad ( Eq . 2 A )$$

### Caspase 3/7 Activity Measurements

Approximately 5,000 MDA-MB-231 cells were plated into white, cell-culture treated, 96-well plates (Corning). At ˜60% confluency, cells were treated with dilutions of LNA-210, Scr-LNA, TGP-210, or TGP-210-RL and then placed under hypoxic or normoxic conditions. After 48 h, caspase 3/7 activity was measured using a Caspase-Glo 3/7 kit (Promega) according to the manufacturer's protocol. Alternatively, MDA-MB-231 cells were transfected with a plasmid overexpressing pre-miR-210 (Genecopoeia; HmiR0167-MR04) using Lipofectamine 2000 (Invitrogen). After 5 h of transfection, cells were plated into white, cell-culture treated 96-well plates (Corning) and then treated as described above. Fold change in Caspase activity was calculated by normalizing treated samples to the untreated samples after subtracting background sample values.

### Measurement of Catalytic Activity

The MDA-MB-231 cells were plated into a 24-well plates (Corning). At ˜80% confluency, the medium was aspirated, and the cell monolayer was washed with 1×DPBS. TGP-210-RL (500 nM) or vehicle (DMSO) was diluted in cell culture medium and added to the cells, which were incubated for 24 h under hypoxic conditions. Cells were removed from hypoxia and then lysed using 250 μL of RNA Lysis Buffer from a Quick-RNA MiniPrep Kit (Zymo Research). A 50 μL aliquot was transferred to black, non-binding surface, half area 96-well plates (Corning). Fractions of untreated cell lysate were combined and used to generate a standard curve of TGP-210-RL in cell lysate, by spiking in known concentrations of TGP-210-RL (1.5625, 3.125, 6.25, 12.5, 25, 50, 100, 200 nM). Fluorescence intensity (Ex: 345 nm, Em: 460 nm) was then measured on a Molecular Devices SpectraMax M5 plate reader. Using the generated standard curve, the concentration of TGP-210-RL in the 50 μL cell lysate aliquot was extrapolated and the amount of TGP-210-RL in pmol in the full 250 μL volume was then calculated.

RNA was extracted from cell lysates using a Quick-RNA MiniPrep Kit (Zymo Research), followed by RT-qPCR proceeded as described above. A standard curve was generated for the pre-miR-210 transcript using in vitro transcribed pre-miR-210 (10 ng, 1 ng, 0.1 ng, 0.01 ng, 0.001 ng, 0.0001 ng, 0 ng) completed with each run to accurately calibrate Cₜ values. The pre-miR-210 transcript was in vitro transcribed as described previously, using the DNA template for miR-210 precursor hairpin RNA (5′—GCAGCCCCTGCC-CACCGCACACTGCGCTGCCCCAGACCCACTGTGCGTGTGACAGCGGCTGATCTG) (SEQ ID NO: 14) and the appropriate Forward (5′—GGCCGGATCCTAATACGACTCACTATAGCAGCCCCTGCCCAC) (SEQ ID NO: 15) and Reverse (5′—CAGATCAGCCGCTGTCAC) (SEQ ID NO: 16) primers. The amount of cleaved pre-miR-210 was then calculated by taking the difference between the pmol of pre-miR-210 in untreated samples and the pmol of pre-miR-210 in TGP-210-RL-treated samples. Catalytic activity, or turnover, was calculated by taking the ratio of the pmol of cleaved pre-miR-210 and the pmol of TGP-210-RL in the sample.

### RNA-Seq

Hypoxic MDA-MB-231 cells were treated as described above for 24 h with TGP-210-RL and total RNA was extracted using a miRNeasy Mini Kit (Qiagen) using an on-column DNase I treatment. A Qubit 2.0 Fluorometer (Invitrogen) and an Agilent Technologies 2100 Bioanalyzer with an RNA nano chip were used to quantify and assess the quality of RNA, respectively. Only samples with RNA Integrity Number >8.0 were used. NEBNext rRNA depletion modules (Catalog #: E6310L, New England Biosciences) were used to deplete rRNA on 500 ng of total RNA, according to manufacturer's instructions. A NEBNext Ultra II Directional RNA kit (Catalog #: E7760, New England Biosciences) was used for library preparation according to the manufacturer's instructions. RNA samples were then chemically fragmented, primed with random hexamers, and reverse transcribed to convert fragmented RNA to first strand cDNA. The RNA template was removed and dUTP was incorporated in place of dTTP, after which the second strand of cDNA was synthesized by end repair and 3′ end adenylation. A hairpin loop adaptor was used to ligate an adaptor A corresponding T nucleotide on the hairpin loop adaptor was used to ligate an adaptor to the double-stranded cDNA. Uracil-specific excision reagent (USER) enzyme was then used to remove the dUTP in the loop, as well as other incorporated U's in the second strand. Final libraries were generated by PCR amplifying adaptor ligated DNA with Illumine barcoding primers, where fragments with both 5′ and 3′ adaptors would be enriched in the final PCR step. Libraries were normalized to 2 nM, validated by a Bioanalyzer DNA chip, pooled equally, and then sequenced on a NextSeq500 v2.5 flow cell (1.8 pM) using paired-end chemistry (2×40 bp). Approximately 20-25 million reads were generated per sample with a base quality score >Q30 (less than 1 error per 1000 bp).

Kallisto was used to quantify transcript abundance, followed by gene-level RNA-Seq differential expression analysis using the Sleuth package in R. TargetScanHuman v7.2 was used to search for predicted microRNA targets for miR-210-3p (only with conserved sites) and for miR-23-3p and miR-107 (top 100 predicted target genes, irrespective of site conservation ranked by cumulative weighted context++ score). The relative % fold change was calculated for each target genes of each microRNA from the RNA-Seq data, using equation 3A (Eq. 3A):

$$Relative \% Fold Change = [ ( \frac{\begin{matrix}Scaled Reads Per Base of TGP - \\ 210 - RL - treated\end{matrix}}{Scaled Reads Per Base of Vehicle - treated} ) - 1 ] \times 100 \qquad ( Eq . 3 A )$$

Significant discrepancy from a binomial distribution of upregulated and downregulated relative % fold change in the target genes were then analyzed using the binomial test function with 95% and 99% confidence in GraphPad Prism 7.

### Chemical Synthesis

### Abbreviations

TGP-210, Targapremir-210; 2′-5′ A, 2′-5′ linked oligoadenylates (Chemgenes); HATU, (1-[Bis(dimethylamino)methylene]-1H-1,2,3-triazolo[4,5-b]pyridinium 3-oxid hexafluorophosphate (Oakwood Chemical); HOAt, 1-Hydroxy-7-azabenzotriazole (Advanced Chem Tech); DIPEA, N,N-Diisopropylethylamine (Sigma-Aldrich); DMSO, dimethyl sulfoxide (EMD); MALDI-TOF, matrix-assisted laser desorption/ionization-time of flight mass spectrometry; HPLC, high performance liquid chromatography; TEAA, triethyl ammonium acetate.

### Synthesis of Targapremir-210 Linked to 2′-5′ Oligoadenylates (TGP-210-2′-5′ Aₙ; n=2−4)

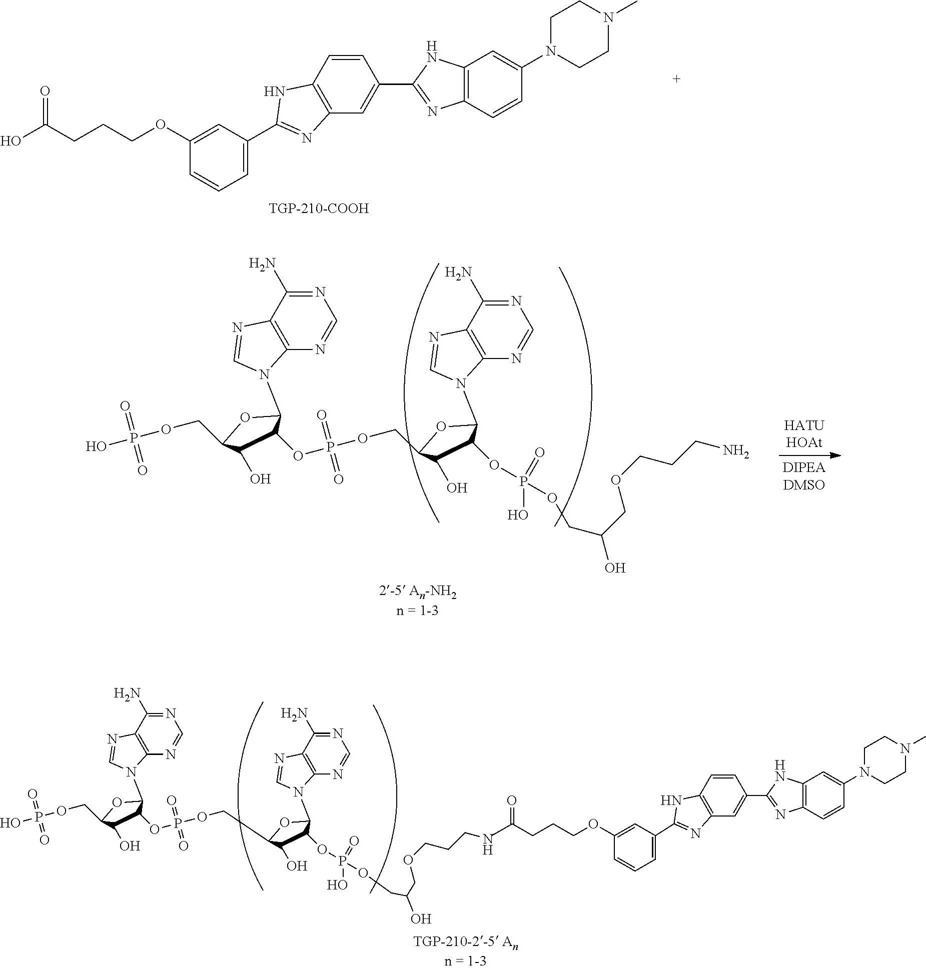

In a 1.6 mL tube, a carboxylic acid derivative of TGP-210 (TGP-210-COOH) (10 μL, 20 mM, 200 nmoles), prepared as previously described, and coupling reagents HATU (2 μL, 100 mM, 200 nmoles) and HOAt (2 μL, 100 mM, 200 nmoles) were added together. The solution was incubated at room temperature for 10 min and then 50 nmol of oligoadenylate amine, (2′-5′ Aₙ-NH₂ where n=2−4) (Chemgenes) and DIPEA (5 μL) were added to the tube. The reaction volume was adjusted with DMSO to 50 μL. The reaction was then incubated at 37° C. with shaking. Reaction progress was monitored by MALDI-TOF using an Applied Biosystems MALDI-TOF/TOF Analyzer 4800 Plus using an α-cyano-4-hydroxycinnamic acid matrix in negative ion mode. The reaction solution was supplemented with additional coupling reagents (200 nmol each) after 8 h as necessary. Upon reaction completion, DMSO was removed under reduced pressure and the mixture was purified by reverse phase HPLC. HPLC purification was performed using a Waters 1525 Binary HPLC pump equipped with a Waters 2487 dual absorbance detector system and a Waters Symmetry C18 5 μm, 4.6×150 mm column using a flow rate of 1 mL/min. A 60 min linear gradient method from 0-100% buffer A to buffer B was used, where buffer B is freshly prepared 50 mM TEAA, pH 7, in water/acetonitrile, 50/50 (v/v) and buffer A is freshly prepared 50 mM TEAA, pH 7, in water. Absorbance was monitored at 254 and 345 nm. Purity was analyzed on an analytical HPLC using the same instrument settings and either a 30 or 60 min using the same linear gradient method.

TGP-210-2′-5′ A₂: Isolated 3.4 nmol (yield=7%); C₅₅H₆₈N₁₇O₂₀P₃ calculated mass: 1378.3961 (M−H)⁻; found: 1378.5798.

TGP-210-2′-5′ A₃: Isolated 18 nmol (yield=36%); C₆₅H₈₀N₂₂O₂₆P₄ calculated mass: 1707.4486 (M−H)⁻; found: 1707.6279.

TGP-210-2′-5′ A₄: Isolated 8.1 nmol (yield=16%); C₇₅H₉₂N₂₇O₃₂P₅ calculated mass: 2036.5012, (M−H)⁻; found: 2036.8623.

### General Synthetic Methods

Abbreviations. DMF, N,N-dimethylformamide; Hex, hexanes; EtOAc, ethyl acetate; DMSO, dimethyl sulfoxide; DCM, dichloromethane; MeOH, methanol; TEA, triethylamine; TFA, trifluoroacetic acid.

Note that compound numbering is independent for each section, so multiple designations of a compound number for intermediates in different synthesis sections is immaterial, the compounds are defined by their structures. The compound numbers for each of the final products are unique for each compound.

### DOCUMENTS CITED

The Encode Project Consortium Nature 2012, 489, 57.
Mandel, M.; Breaker, R. R. Nat Rev Mol Cell Biol 2004, 5, 451.
Cech, Thomas R.; Steitz, Joan A. Cell 2014, 157, 77.
Moore, M. J. Science 2005, 309, 1514.
Sakamoto, K. M.; Kim, K. B.; Kumagai, A.; Mercurio, F.; Crews, C. M.; Deshaies, R. J. Proc Natl Acad Sci USA 2001, 98, 8554.
Winter, G. E.; Buckley, D. L.; Paulk, J.; Roberts, J. M.; Souza, A.; Dhe-Paganon, S.; Bradner, J. E. Science 2015, 348, 1376.
Silverman, R. H. J Virol 2007, 81, 12720.
Torrence, P. F.; Xiao, W.; Li, G.; Cramer, H.; Player, M. R.; Silverman, R. H. Antisense Nucleic Acid Drug Dev 1997, 7, 203.
Velagapudi, S. P.; Cameron, M. D.; Haga, C. L.; Rosenberg, L. H.; Lafitte, M.; Duckett, D. R.; Phinney, D. G.; Disney, M. D. Proc Natl Acad Sci USA 2016, 113, 5898.
Floyd-Smith, G.; Slattery, E.; Lengyel, P. Science 1981, 212, 1030.
Al-Ahmadi, W.; Al-Ghamdi, M.; al-Haj, L.; Al-Mohanna, F. A.; Silverman, R. H.; Khabar, K. S. A. Oncogene 2009, 28, 1782.
Lin, H.; Dai, T.; Xiong, H.; Zhao, X.; Chen, X.; Yu, C.; Li, J.; Wang, X.; Song, L. PLOS ONE 2010, 5, e15797.
Fu, Z.; Tindall, D. J. Oncogene 2008, 27, 2312.
Bondeson, D. P.; Mares, A.; Smith, I. E. D.; Ko, E.; Campos, S.; Miah, A. H.; Mulholland, K. E.; Routly, N.; Buckley, D. L.; Gustafson, J. L.; Zinn, N.; Grandi, P.; Shimamura, S.; Bergamini, G. Faelth-Savitski, M.; Bantscheff, M.; Cox, C.; Gordon, D. A.; Willard, R. R.; Flanagan, J. J.; Casillas, L. N.; Votta, B. J.; den Besten, W.; Famm, K.; Kruidenier, L.; Carter, P. S.; Harling, J. D.; Churcher, I.; Crews, C. M. Nat Chem Biol 2015, 11, 611.
Thakur, C. S.; Jha, B. K.; Dong, B.; Das Gupta, J.; Silverman, K. M.; Mao, H.; Sawai, H.; Nakamura, A. O,; Banerjee, A. K.; Gudkov, A.; Silverman, R. H. Proc. Natl. Acad. Sci. U.S.A. 2007, 104, 9585.
Velagapudi, S. P.; Cameron, M. D.; Haga, C. L.; Rosenberg, L. H.; Lafitte, M.; Duckett, D. R.; Phinney, D. G.; Disney, M. D. Proc. Natl. Acad. Sci. 2016, 113, 5898.
Özçubukçu, S.; Ozkal, E.; Jimeno, C.; Pericàs, M. A. Org. Lett. 2009, 11, 4680.
Pushechnikov, A.; Lee, M. M.; Childs-Disney, J. L.; Sobczak, K.; French, J. M.; Thornton, C. A.; Disney, M. D. J. Am. Chem. Soc. 2009, 131, 9767.
Dong, B.; Silverman, R. H. J. Biol. Chem. 1997, 272, 22236.
Velagapudi, S. P.; Gallo, S. M.; Disney, M. D. Nat. Chem. Biol. 2014, 10, 291.
Dong, B.; Niwa, M.; Walter, P.; Silverman, R. H. RNA 2001, 7, 361.

1. A compound, or a pharmaceutically acceptable salt thereof, having a structure of Formula I: 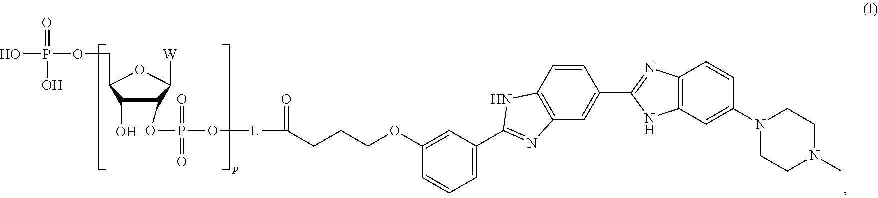
wherein
W is a nucleobase;
L is a linker moiety, and
p is 1 to 5.

2. The compound or salt of claim 1, wherein L comprises 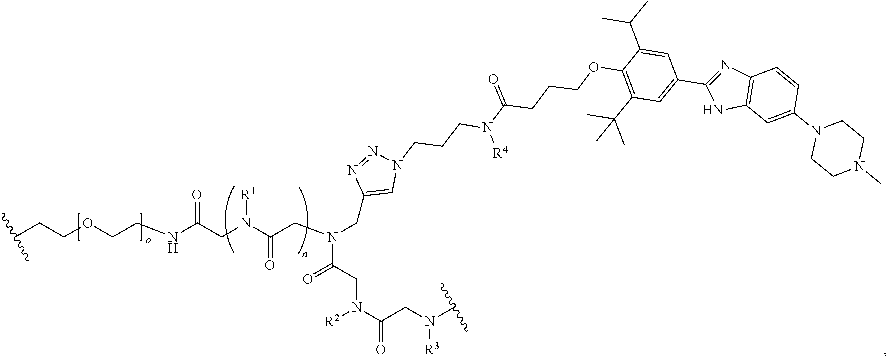
wherein:
R¹, R², R³, and R⁴ are each independently H or C₁₋₆ alkyl;
n is 0 to 9; and
o is 1 to 5.

3. The compound or salt of claim 1 wherein L is C₂₋₆ alkylene-O—C₂₋₆ alkylene-NR³ and one or both of the C₂₋₆ alkylenes is optionally substituted by one or two hydroxyl groups.

4. The compound or salt of claim 2, wherein W is adenine.

5. The compound or salt of claim 3, wherein W is adenine.

6. The compound or salt of claim 4, wherein at least one of R¹, R², R³, R⁴ is C₁₋₆ alkyl.

7. (canceled)

8. (canceled)

9. The compound or salt of claim 6, wherein all of R¹, R² and R³ are C₁₋₆ alkyl.

10. The compound or salt of claim 9, wherein R⁴ is H.

11. The compound or salt of claim 5, herein L is

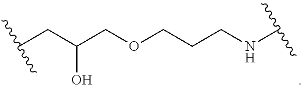 [SMILES: *CC(O)COCCCN*]

12. The compound or salt of claim 9, wherein L is

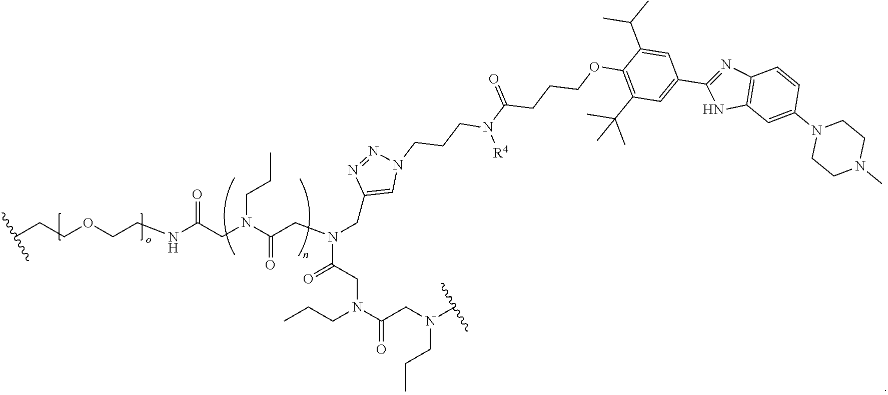

13. (canceled)

14. The compound or salt of claim 12, wherein p is 4.

15. (canceled)

16. (canceled)

17. The compound or salt of claim 14 wherein o is 2.

18. The compound or salt of claim 1, having the structure of Formula (Ia):

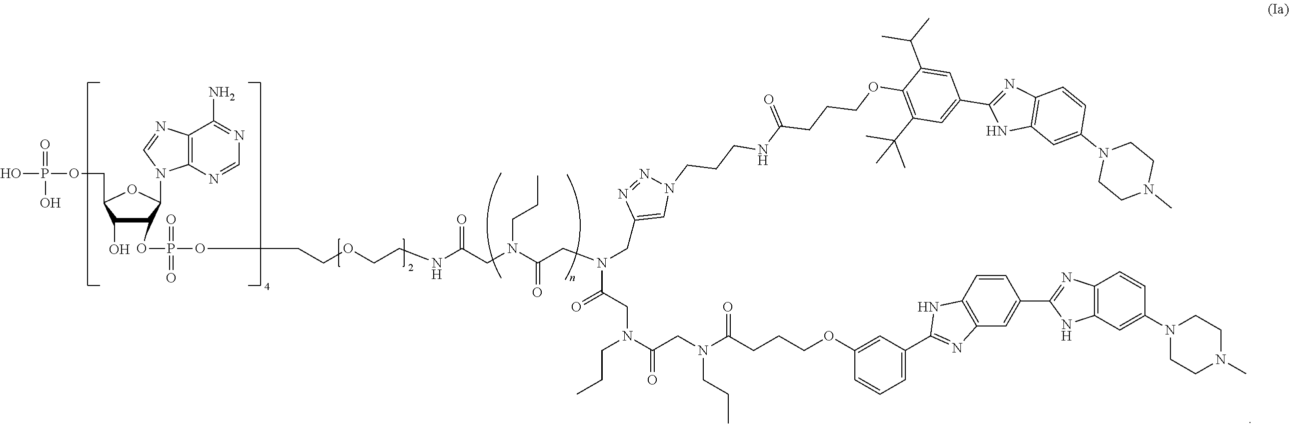

19. The compound or salt of claim 18, wherein n is 0, 3, 6, or 9.

20. (canceled)

21. (canceled)

22. (canceled)

23. A compound of Table A, or a pharmaceutically acceptable salt thereof.

24. A method of cleaving a pri-miR-96 hairpin RNA nucleic acid or pre-miR-210 precursor hairpin RNA nucleic acid inside a cancer cell comprising contacting the nucleic acid with an effective amount of the compound or salt of claim 1.

25. The method of claim 24, wherein the cancer cell is a breast cancer cell.

26. A method of cleaving a pri-miR-96 hairpin RNA nucleic acid inside a cancer cell comprising contacting the nucleic acid with an effective amount of the compound or salt of claim 18.

27. (canceled)

28. A method of cleaving a pre-miR-210 hairpin RNA nucleic acid inside a cell comprising contacting the nucleic acid with an effective amount of the compound or salt of claim 11 and p is 4.

29. The method of claim 28, wherein the cancer cell is a breast cancer cell.

30. (canceled)

31. (canceled)

32. A method of treating a disease or disorder involving cancer comprising administering to a patient in need thereof a therapeutically effective amount of the compound or salt of claim 1.

33. (canceled)

34. The method of claim 32, wherein the cancer is breast cancer or triple negative breast cancer.

35. (canceled)

36. The method of claim 34, wherein administering the compound or salt de-represses pro-apoptotic FOXO1 transcription factor and triggers apoptosis in the breast cancer cells or triple negative breast cancer cells.

37. (canceled)

38. The method of claim 34, wherein administering the compound or salt de-represses GPD1L protein, which binds to prolyl hydroxylase (PHD) to promote hyperhydroxylation of hypoxia inducible factor 1-alpha (HIF1α), mediating HIF1α degradation by the proteasome, and triggering apoptosis in a breast cancer cell.

39. (canceled)

40. The method of claim 38, wherein the therapeutically effective amount of the compound or salt does not trigger apoptosis in a healthy breast cell.

41. (canceled)

42. A method of cleaving RNA comprising contacting the RNA within a cancer cell with a compound, or pharmaceutically acceptable salt thereof, having a structure of A₂₋₄-linker-Ht, wherein
A is adenine nucleotide,
linker comprises 5 to 150 carbon atoms optionally interrupted with 1 to 20 heteroatoms individually selected from N, O and S, and
Ht is an RNA-targeting group.

43. The method of claim 42, wherein Ht comprises

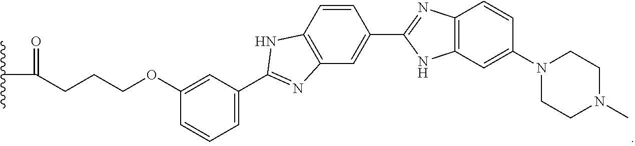 [SMILES: *C(=O)CCCOc1cccc(-c2nc3cc(-c4nc5ccc(N6CCN(C)CC6)cc5[nH]4)ccc3[nH]2)c1]

44. The method of claim 43, wherein the compound or salt comprises A₄-linker-Ht.


SEQUENCE LISTING (22 sequences)

SEQ ID NO: 1 - DNA, 36 bp, Artificial Sequence
  Synthetic Polynucleotide
  misc_feature (1)..(1): 6FAM
uuaucaaauucuuauuugccccauuuuuuugguuua

SEQ ID NO: 2 - DNA, 78 bp, Artificial Sequence
  Synthetic Polynucleotide
uggccgauuuuggcacuagcacauuuuugcuugugucucuccgcucugagcaaucaugugcagugccaauaugggaaa

SEQ ID NO: 3 - DNA, 81 bp, Artificial Sequence
  Synthetic Polynucleotide
gggtggccgattttggcactagcacatttttgcttgtgtctctccgctctgagcaatcatgtgcagtgccaatatgggaaa

SEQ ID NO: 4 - DNA, 44 bp, Artificial Sequence
  Synthetic Polynucleotide
ggccggatcctaatacgactcactatagggtggccgattttggc

SEQ ID NO: 5 - DNA, 15 bp, Artificial Sequence
  Synthetic Polynucleotide
tttcccatattggca

SEQ ID NO: 6 - DNA, 60 bp, Artificial Sequence
  Synthetic Polynucleotide
gggagagggtttaatccgattttggtacgaaagtaccaatatgggattggatccgcaagg

SEQ ID NO: 7 - DNA, 60 bp, Artificial Sequence
  Synthetic Polynucleotide
gggagagggtttaatccgattttggtacgaaagtaccaaaatcggattggatccgcaagg

SEQ ID NO: 8 - DNA, 42 bp, Artificial Sequence
  Synthetic Polynucleotide
ggccgaattctaatacgactcactatagggagagggtttaat

SEQ ID NO: 9 - DNA, 15 bp, Artificial Sequence
  Synthetic Polynucleotide
ccttgcggatccaat

SEQ ID NO: 10 - DNA, 58 bp, Artificial Sequence
  Synthetic Polynucleotide
  misc_feature (1)..(1): 6FAM
agccccugcccaccgcacacugcgcugccccagacccacugugcgugugacagcggcu

SEQ ID NO: 11 - DNA, 32 bp, Artificial Sequence
  Synthetic Polynucleotide
  misc_feature (1)..(1): Cy5
cgcacacugcgcugccccagacccacugugcg

SEQ ID NO: 12 - DNA, 32 bp, Artificial Sequence
  Synthetic Polynucleotide
  misc_feature (1)..(1): Cy5
cgcacagugcgcugccccagacccacugugcg

SEQ ID NO: 13 - DNA, 28 bp, Artificial Sequence
  Synthetic Polynucleotide
  misc_feature (1)..(1): Cy5
cgcgaattcgcgttttcgcgaattcgcg

SEQ ID NO: 14 - DNA, 66 bp, Artificial Sequence
  Synthetic Polynucleotide
gcagcccctgcccaccgcacactgcgctgccccagacccactgtgcgtgtgacagcggctgatctg

SEQ ID NO: 15 - DNA, 42 bp, Artificial Sequence
  Synthetic Polynucleotide
ggccggatcctaatacgactcactatagcagcccctgcccac

SEQ ID NO: 16 - DNA, 18 bp, Artificial Sequence
  Synthetic Polynucleotide
cagatcagccgctgtcac

SEQ ID NO: 17 - DNA, 81 bp, Artificial Sequence
  Synthetic Polynucleotide
ggguggccgauuuuggcacuagcacauuuuugcuugugucucuccgcucugagcaaucaugugcagugccaauaugggaaa

SEQ ID NO: 18 - DNA, 23 bp, Artificial Sequence
  Synthetic Polynucleotide
acugugcgugugacagcggcuga

SEQ ID NO: 19 - DNA, 62 bp, Artificial Sequence
  Synthetic Polynucleotide
ggagccccugcccaccgcacacugcgcugccccagacccacugugcgugugacagcggcuga

SEQ ID NO: 20 - DNA, 50 bp, Artificial Sequence
  Synthetic Polynucleotide
ccugcccaccgcacacugcgcugccccagacccacugugcgugugacagc

SEQ ID NO: 21 - DNA, 32 bp, Artificial Sequence
  Synthetic Polynucleotide
cgcacacugcgcugccccagacccagugugcg

SEQ ID NO: 22 - DNA, 60 bp, Artificial Sequence
  Synthetic Polynucleotide
agccccugcccaccgcacacugcgcugccccagacccacugugcgugugacagcggcuga